(19) United States
(12) Patent Application Publication
(10) Pub. No.: US 2006/0269528 A1
(43) Pub. Date: Nov. 30, 2006

(54) Production detection and use of transformant cells

(76) Inventors: Steven Gary Kendrew, Cambridge (GB); Hrvoje Petkovic, Cambridge (GB); Peter Francis Leadlay, Cambridge (GB); Hamish Alastair Irvine McArthur, Mystic, CT (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/492,099

(22) PCT Filed: Oct. 17, 2002
(86) PCT No.: PCT/GB02/04695

§ 371 Date: Sep. 13, 2004

(30) Foreign Application Priority Data
Oct. 17, 2002 (GB) 0125043.0

(51) Int. Cl. A61K 48/00; A61K 39/02; C12N 1/21; C12N 1/18; C12N 5/08; C12N 15/74

(52) U.S. Cl. 424/93.21; 435/455; 435/366; 435/252.3; 435/254.2; 424/200.1; 435/471

(57) ABSTRACT

A bioactive compound, or family of compounds, is biosynthesised by a cell using a plurality of enzymic activities. Host cells are provided that substantially lack the bioactivity, but possess at least a first one of the enzymic activities (or nucleic acid encoding a corresponding enzyme). The cells are transformed with nucleic acid expressible to provide at least a second enzymic activity which enables the cells to produce a bioactive compound. A family of cells may be transformed with a multiplicity of different nucleic acids, leading to a library of cells including cells producing different bioactive product, and cells not producing bioactive products. Transformed cells may be screened for bioactivity. Active cells may be isolated and cultured, and bioactive compounds may be isolated.

The present invention relates to the production, detection and use of transformant cells.

### BACKGROUND

Over the past 50 years pharmaceutical companies have used screening of natural products produced by bacteria, fungi, invertebrates and plants as a fruitful source of many important drugs. Many of the chemicals produced by these organisms are biologically active and a relatively high percentage has successfully completed extensive clinical testing regimes and are marketed commercially. Indeed, if we ignore biologicals such as vaccines and monoclonal antibodies, over 60% of approved and pre-NDA candidate drugs are either natural products or related to them. These include, in human health usage; antibacterials such as erythromycin, tetracycline, penicillin, pristinamycin and streptomycin, antifungals such as amphotericin and nystatin, immunosuppressants such as FK506, cyclosporin and rapamycin, anticancers such as taxol, and doxorubicin, antivirals such as lamivudine and anticholesterol agents such as lovastatin and pravistatin; in agricultural use; insecticidals such as spinosyn, antihelminthics such as avermectin, bioherbicides such as bialaphos and anticoccidial growth promoters such as monensin and tylosin. While companies continue to screen their culture collections and natural product libraries for novel biological activities there is a sense of diminishing return particularly in anti-infective discovery (Biotechnology of Antibiotics (1997), Ed. W. R Strohl). Many of the new compounds displaying activity possess broadly similar chemical structures and belong to existing classes of natural products without a significantly different activity spectrum (Bindseil K. U. et al. (2001) DDT, 6, 840). There is a need to discover greater numbers of natural products or products based on naturally occurring molecules as this will lead to the discovery of novel bioactivities (Abel, U. et al. (2002) Curr. Opin. Chem Biol, 6, 453-458).

The emergence of bacterial strains resistant to current antibiotic regimes has increased the need to produce a greater range of compounds for testing. The speed at which pathogenic bacteria have acquired this resistance has vastly outpaced the development and testing of commercial alternatives. At the same time advances in our understanding of drug-protein targets and cellular processes has resulted in exponential increases in the numbers and methods of screens (across many different bioactivities) a pharmaceutical company has at its disposal. Typical screening programs will include either protein targets or whole cell assays or whole organism assays. In addition to drug discovery screening programs, similar technologies have also been developed for a number of other commercially useful products including agrochemicals or proteins. Much of this screening research has been driven by investment in combinatorial chemistry programs, many of which generate hundreds of thousands of compounds in a short space of time. To date, however, relatively few of these compounds have resulted in a marketable entity.

It has been hard for traditional approaches to generate natural product diversity to keep pace with the changes; while combinatorial chemistry programs have produced large numbers of compounds; natural product discovery groups have failed to supply the numbers required to feed the high-throughput screening apparatus. While it has always been recognised that greater proportions of natural product candidates will display biological activity the scientists have not managed to produce the numbers in the same ways as the combinatorial chemists. There is a need to develop methods to generate larger numbers of compounds in natural product libraries to utilise the powerful pharmaceutical screening systems developed by these companies. Furthermore, since natural products may represent a template on which to perform combinatorial chemistry they can add a further degree of diversity to an existing combinatorial chemistry program.

One approach to the generation of natural product diversity has been to study an ever-widening range of sources both geographical and biological. For example, the total number of natural products produced by plants is estimated to be in excess of 500,000 of which only a fraction has been isolated. Compounds from marine organisms, both bacterial and invertebrate, are also poorly represented. However, finding the conditions for compound production from these systems has been difficult and alternative approaches are needed in parallel. Manipulating media conditions to trigger production of a greater range of substances from existing organisms is another technique used to generate greater compound numbers.

Another key focus has been on the study of ‘unculturable’ or ‘environmental’ (including marine) organisms in the belief that the molecular differences that determine why these organisms cannot be cultivated in the laboratory may also indicate potential to produce different classes of compounds. These compounds might display different ranges of biological activity to those from more conventional backgrounds. Typically, the process involves extracting large sections of DNA or amplifying portions of DNA extracted from soil or other environmental or unusual samples and expressing them in a host that can be cultivated. These techniques have in some cases overcome the original organism's regulatory cascades that control compound production.

However, advances in biochemistry and molecular biology, and in particular, advances in our understanding of the in vivo biosynthetic processes that lead to natural product production have led to a number of novel drug discovery approaches. Cloning and sequencing of genes that are responsible for the production of secondary metabolite biosynthetic enzymes and the discovery that in many cases these enzymes are clustered together has vastly influenced our ability to manipulate the pathways to produce novel structures and hence increased diversity in both structure and function. Much of this work has been centred on polyketide or nonribosomal peptide biosynthesis; this partially reflects the commercial interest in these compounds, which apart from their wide range of biological activities continue to provide strong revenue streams for interested parties. However, it is clear that principles and methodologies developed by researchers in these particular fields are equally applicable to other natural product biosynthetic systems whether from prokaryotic or eukaryotic microorganisms or higher organisms.

There is a clear need to develop technologies that can utilise the genetic understanding and methodologies that lead to novel structural analogues or diversity of natural products and can transfer the molecular diversity so produced into an easily screenable format that can be utilised by screening programs and high-throughput apparatus of pharmaceutical companies. While we use the manipulation and screening of polyketides as illustrative of the principles behind the present invention it is not intended to limit the technology to these systems. The disclosed methodology is applicable to any biosynthetic pathway from any species.

Polyketides are a large and structurally diverse class of natural products that includes many compounds possessing antibiotic or other pharmacological properties, such as erythromycin, tetracyclines, rapamycin, avermectin, monensin, epothilones and FK506. In particular, polyketides are abundantly produced by soil organisms such as Streptomyces and related actinomycete bacteria. They are synthesised by the repeated stepwise condensation of acylthioesters in a manner analogous to that of fatty acid biosynthesis. The structural diversity found among natural polyketides arises in part from the selection of (usually) acetate (malonyl-CoA) or propionate (methylmalonyl-CoA) as “starter” or “extender” units (although one of a variety of other types of unit may occasionally be selected); as well as from the differing degree of processing of the β-keto group formed after each condensation. Examples of processing steps include reduction to β-hydroxyacyl-, reduction followed by dehydration to 2-enoyl-, and complete reduction to the saturated acyl-thioester. The stereochemical outcome of these processing steps is also specified for each cycle of chain extension. Methylation at the α-carbon or at the oxygen substituent is also sometimes observed.

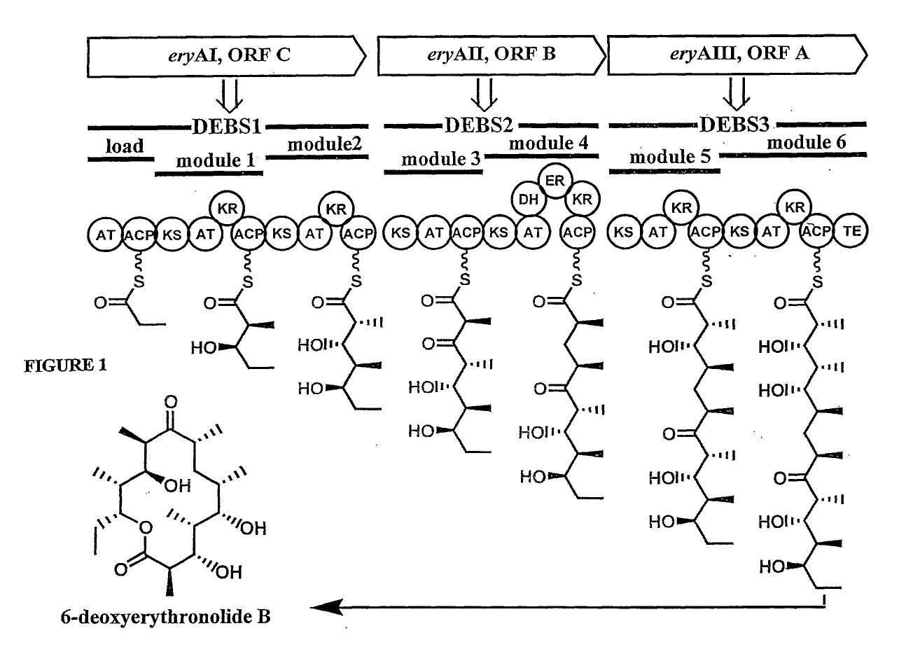

The biosynthesis of polyketides is performed by a group of chain-forming enzymes known as polyketide synthases. The term “polyketide synthase” (PKS) as used herein refers to a complex of enzyme activities responsible for the biosynthesis of polyketides. These enzyme activities include β-ketoacyl ACP synthase (KS), acyltransferase (AT), acyl carrier protein (ACP), β-ketoreductase (KR), dehydratase (DH), enoylreductase (ER) and thioesterase (TE) but are not limited to these activities. Two broad classes of polyketide synthase (PKS) have been described in actinomycetes. One class, named Type I PKSs, represented by the PKSs for the macrolides erythromycin, oleandomycin, avermectin, and rapamycin and by the PKS for the polyether monensin, are multidomain proteins comprising of a different set or “module” of enzymes for each cycle of polyketide chain extension. For examples see FIG. 1 (Cortés, J. et al. Nature (1990) 348:176-178; Donadio, S. et al. Science (1991) 2523:675-679; Swan, D. G. et al. Mol. Gen. Genet. (1994) 242:358-362; MacNeil, D. J. et al. Gene (1992) 115:119-125; Schwecke, T. et al. Proc. Natl. Acad. Sci. USA (1995) 92:7839-7843; also Patent application WO98/01546). The genes encoding numerous Type I PKSs have been sequenced and these sequences disclosed in publicly available DNA and protein sequence databases including Genbank, Swissprot and EMBL. For example, the sequences are available for the PKSs governing the synthesis of, respectively, erythromycin (Cortes, J. et al. Nature (1990) 348:176-178); accession number X62569, Donadio, S. et al. Science (1991) 252:675-679; accession number M63677 or U.S. Pat. No. 5,824,513); rapamycin (Schwecke, T. et al. Proc. Natl. Acad. Sci. (1995) 92:7839-7843; accession number X86780); rifamycin (August, P. et al. Chem. Biol. (1998) 5:69-79; accession number AF040570) and tylosin (Eli Lilly, accession numer U78289), among many others.

The second class of PKS, named Type II PKSs, is represented by the synthases for aromatic compounds. Type II PKSs contain only a single set of enzymatic activities for chain extension and these are re-used as appropriate in successive cycles (Bibb, M. J. et al. EMBO J. (1989) 8:2727-2736; Sherman, D. H. et al. EMBO J. (1989) 8:2717-2725; Fernandez-Moreno, M. A. et al. J. Biol. Chem. (1992) 267:19278-19290). The “extender” units for the Type II PKSs are usually acetate (malonyl-CoA) units, and the presence of specific cyclases dictates the preferred pathway for cyclisation of the completed chain into an aromatic product (Hutchinson, C. R. and Fujii, I. Annu. Rev. Microbiol. (1995) 49:201-238). Occasionally, unusual starter units are incorporated by Type II PKS, particularly in the biosynthesis of oxytetracycline, frenolicin and daunorubicin and in these cases a separate AT is used to transfer the starter unit to the PKS. Hybrid polyketides have been obtained by the introduction of cloned Type II PKS gene-containing DNA into another strain containing a different Type II PKS gene cluster, for example by introduction of DNA derived from the gene cluster for actinorhodin, a blue-pigmented polyketide from Streptomyces coelicolor, into an anthraquinone polyketide-producing strain of Streptomyces galileus (Bartel, P. L. et al. J. Bacteriol. (1990) 172:4816-4826).

Other types of polyketide synthases exist but are less well studied, but it appears many similar biosynthetic mechanistic principles apply and they are likely to be equally amenable to genetic manipulation. These include the fungal PKS such as the 6-methylsalicylic acid synthase of yeast or the lovastatin PKS of Aspergillus terreus which consist of a single multi-domain polypeptide which include most of the activities required for the synthesis of the polyketide portion of these molecules (Hutchinson C. R. and Fujii I. Annu. Rev. Microbiol. (1995) 49:201-238). Type II Fungal PKS are also known. Also known are type III polyketide synthases, or chalcone synthases, which are responsible for the biosynthesis of many polyketides in plants among other organisms, including bacteria. Recently, the analogy between polyketide and fatty acid biosynthesis has been further strengthened with the discovery of PKS-like fatty acid synthases responsible for unsaturated fatty acid biosynthesis in a microbial and a eukaryotic organism (Metz J. G. et al (2001) Science, 293, 290-293). Further types PKS have been described, including Liu, W. et al. (2002) Science, 297, 1170, Alhert J. et al. (2002) Science 297, 1173 and Kwon, H.-J. et al. Science 297, 1327.

Non-ribosomal peptide synthases (NRPS) that produce the diverse array of peptide antibiotics such as gramicidin, pristinamycin and bacitracin, and the immunosuppressant cyclosporin are conceptually similar to the Type I polyketide synthases. They comprise large multidomain proteins sharing a common mode of biosynthesis through the thiotemplate mechanism. As for Type I PKS, they are arranged in modules, each module responsible for the activation, condensation and processing (e.g. epimerisation) of one amino acid of the peptide. Each module corresponds with the sequence of amino acids on the final peptide product. Manipulation of the modules by genetic engineering of the NRPS in the same manner as used for Type I PKS's has resulted in alteration of the final peptide produced (Marahiel M., et al. (1997) Chem. Rev. 97, 2651-2673, Mootz, H. D. et al. (2000) PNAS, 97, 5848-5853) among others. NRPS appear in a wide range of organisms both prokaryotic and eukaryotic. Mixed NRPS-PKS systems (ie those containing components of both polyketide and non-ribosomal peptides) exist including epothilone, bleomycin and one of the paradigm gene clusters rapamycin.

Other biosynthetic systems include alkaloids, terpenes, aminoglycosides, shikimic acid derivatives, polysaccharides, flavones and other flavonoids, nitrogen containing compounds such as indoles and pyrroles, steroids and other hormones, anthraquinones, lignans, coumarins, stilbenes, depsipeptides and peptides and proteins among many others. These and other natural product biosynthetic systems are equally amenable to the use of the technology described herein.

Although numbers of therapeutically important polyketides have been identified, there remains a need to obtain novel polyketides that have enhanced properties or possess completely novel bioactivity. The complex polyketides produced by type I PKSs are particularly valuable, in that they include compounds with known utility as anthelmintics, insecticides, immunosuppressants, antifungal or antibacterial agents. Because of their structural complexity, such novel polyketides are not readily obtainable by total chemical synthesis or by chemical modification of known polyketides. Particular changes that are desired are changes to the carbon skeleton by altering the nature of the starter/extender unit incorporated, changes to the oxidation level of the β-keto carbon and therefore altering the pattern and or stereochemistry of oxygen substituents by altering the series of reductive steps that occur after chain extension and, changes to the post PKS “tailoring” steps which generally comprise, for example, hydroxylation, methylation or glycosylation of the polyketide molecule.

Several methods have been described to manipulate Type I PKS to generate novel structural analogues of the original natural product. The length of the polyketide chain formed by the synthase has been altered, in the case of erythromycin biosynthesis, by specific relocation using genetic engineering of the enzymatic domain of the erythromycin-producing PKS that contains the chain-releasing thioesterase/cyclase activity (Cortés, J. et al. Science (1995) 268:1487-1489; Kao, C. M. et al. J. Am. Chem. Soc. (1995) 117:9105-9106).

In-frame deletion of the DNA encoding part of the ketoreductase domain in module 5 of the erythromycin-producing PKS (also known as 6-deoxyerythronolide B synthase, DEBS) has been shown to lead to the formation of erythromycin analogues 5,6-dideoxy-3-Omycarosyl-5-oxoerythronolide B and 5,6-dideoxy-5-oxoerythronolide B (Donadio, S. et al. Science (1991) 252:675-679). Likewise, alteration of active site residues in the enoylreductase domain of module 4 in DEBS, by genetic engineering of the corresponding PKS-encoding DNA and its introduction into Saccharopolyspora erythraea, led to the production of 6,7-anhydroerythromycin C (Donadio, S. et al. Proc Natl. Acad. Sci. USA (1993) 90:7119-7123). Many other examples of engineering of a polyketide synthase to give novel polyketide structures are described in publically available literature.

Patent applications WO98/01546 and U.S. Pat. No. 5,824,513 describe further methods of manipulating Type I PKS enzymes. Patent application WO/00/01827 describes further methods of manipulating a PKS to change the oxidation state of the β-carbon. Substituting the reductive domain of module 2 of the erythromycin-producing PKS with domains derived from rapamycin PKS modules 10 and 13 led to the formation of C10-C11 olefin-erythromycin A and C10-C11 dihydroerythromycin A respectively. Patent U.S. Pat. No. 6,200,813 describes methods of manipulating the polyketide backbone. Patent application WO00/00500 describes novel methods for making macrolides with specific starter units. Patent WO02/14482 describes methods of altering the substrate specificity of the AT domain. U.S. Pat. No. 5,998,194, and Gaisser S. et al. (2000) Mol. Microbiol, 36, 391-401 among many others disclose methods of manipulating the various enzymes that process the completed polyketide chain. Similar manipulations of the biochemical pathway have been described for the nonribosomal peptide synthase systems.

Recombinant production of PKS and subsequent production of polyketides has been illustrated; many examples are available in heterologous or native systems. In early work each of the DEBS proteins of the erythromycin PKS were expressed in E. coli although it is likely the ACP was inactive in this case (Roberts G. A. et al. Eur J. Biochem, 214, 305-311). Related methods have been used more recently to produce the erythromycin aglycone 6DEB in engineered E. coli cells (Pfiefer, B. A. et al. (2001) Science 291, 1790-1792) by co-expression of all three DEBS proteins with an holo-ACP synthase to activate the ACP and additionally engineering the cells to provide simple precursor molecules. U.S. Pat. No. 6,033,883 describes methods for production in bacteria and yeast. Kennedy et al. (1999) (Science, 284, 1368-72) describe heterologous production of the lovastatin PKS and production of biosynthetic intermediates in Aspergillus nidulans.

A host vector system has been developed that allows directed mutation and expression of cloned PKS genes (McDaniel et al. 1993, Science 262:1546-1550; Kao et al 1994 Science 265:509-512). This vector system relies on the use of the actI/actII-orf4 promoter system and is used in S. coelicolor. This vector system has been used to develop more efficient methods of producing polyketides (U.S. Pat. No. 5,672,491, WO95/08548, U.S. Pat. No. 5,712,146, U.S. Pat. No. 5,830,750, U.S. Pat. No. 5,843,718, U.S. Pat. No. 5,962,290, U.S. Pat. No. 6,022,731, U.S. Pat. No. 6,077,696), particularly type II polyketides. This system has been used for Type I polyketide production, however it is concerned only with the production of the (usually inactive) polyketide backbone.

The actI/actII-orf4 promoter has also been used heterologously in Saccharopolyspora erythraea as described in WO98/01546 and Rowe et al. (1998) Gene, 216, 215-223.

Patents WO 98/49315, PCT/US98/08792 and WO 00/63361 and Xue, Q. et al. (1999) PNAS, 96, 11740-11745 describe novel methods to manipulate polyketide synthase genes to produce combinatorial libraries of polyketides, again concerned solely with production of the inactive polyketide backbones. A particular disadvantage of these expression methodologies is that in most cases the isolated PKS products have to be further transformed to yield biologically active species.

A number of methods have been disclosed for the production and screening of genetically transformed organisms. Patent WO 99/67374 and Sosio, M. F. et al. (2000) Nat. Biotech. 18, 343-345 is particularly concerned with the movement of the entire gene cluster responsible for the production of a natural product from the natural host to an alternative organism. This discloses a method of producing and manipulating natural products by transferring the ability to produce a secondary metabolite from the original producer to another production host that has desirable characteristics, for example ease of transformation or cultivation. The invention is particularly concerned with the use of Bacterial Artificial Chromosomes (BACs) (termed ESACs, E. coli-Streptomyces Artificial Chromosomes) that can be shuttled between a convenient neutral cloning host and a production host. These vectors have the ability to contain large (e.g. entire biosynthetic cluster) pieces of DNA. Libraries of high molecular weight genomic DNA are produced in these chromosomes and shuttled between E. coli and the Streptomyces production host selecting for clones directing synthesis of the natural product. Alternatively, a large segment of DNA that directs synthesis of a natural product is reconstructed from the genome of the donor organism utilising the artificial chromosomes. U.S. Pat. No. 5,824,485, U.S. Pat. No. 5,783,431, and U.S. Pat. No. 6,242,211 (Chromaxome) describe a drug discovery system for generating and screening molecular diversity. The patents describe methods of generating and manipulating gene expression libraries. They are primarily concerned with the capture and assay of essentially random portions of genetic material of organisms that are prospective sources of drug leads, particularly those that cannot be recovered in substantial amounts in nature or be cultured in the laboratory (‘unculturables’). Methods of biasing or generating diversity in these libraries by for example random concatenation or recombination are also disclosed. U.S. Pat. No. 5,958,672, U.S. Pat. No. 5,939,250, U.S. Pat. No. 6,057,103, U.S. Pat. No. 6,030,779 and U.S. Pat. No. 6,174,673 disclose similar techniques for library construction, screening and manipulating DNA again primarily genomic DNA derived from unculturable organisms. U.S. Pat. No. 5,837,470, U.S. Pat. No. 5,773,221 and U.S. Pat. No. 5,908,765 refer to methods of recovering a biological molecule from a recombinant organism made using environmental samples. Seow et al. (1997) J. Bact. 179, 7360-8 discusses methods of cloning and expressing random PKS genes from soil DNA.

These methods rely on what can be termed “superhost” methodology, in which an entire biosynthetic cluster is transferred to an alternative, sometimes genetically “cleaned” (by removal of alternative clusters for example) strain for production. The use of such a strain greatly aids the expression of entire pathways from unculturable organisms or reduces production of competing compounds. The host cell is simply used as a ‘bag’ in which the introduced genetic material can function, the cell might provide cofactors or simple precursor molecules from primary metabolic pathways but does not participate to a great extent in the biosynthesis of the active molecule.

Disclosed techniques utilising random cloning of DNA into a host cell have a disadvantage. The size of natural product clusters often exceeds the natural limits of current cloning/expression vectors, hence the development of systems utilising bacterial artificial chromosomes that can carry larger amounts of DNA. However, many of the desired biosynthetic pathway manipulations utilising technology developed in the field are not possible in such vectors as their overall large size inhibits facile genetic manipulation of the pathways, because for example, unique restriction enzyme sites are rare. In addition, some biosynthetic pathways are not entirely clustered precluding reconstitution from a single piece of genomic DNA in a host cell. Hence, a drug discovery or screening methodology that produces active products but does not rely on the insertion of an entire biosynthetic pathway on a single vector is of considerable benefit.

### SUMMARY OF THE INVENTION

The present invention relates to a method for preparing and detecting bioactive materials, which may be novel, utilising genetic information from biosynthetic pathways.

To produce a cell which has a desired bioactivity (because it expresses a bioactive material), a cell is selected or prepared which does not have the desired bioactivity (or at least, not to a substantial extent) but does have at least a first activity that can contribute to the generation of the desired bioactivity. This first activity may result from expression of nucleic acid possessed by the cell. The first activity may be, for example, a glycosylation activity. Genetic material encoding at least a second activity is used to transform the cell so that it acquires the second activity. The second activity may lead, directly or indirectly, to the production of a material which is a substrate for the first activity, leading (directly or indirectly) to said bioactive material. Cells may be screened directly for the bioactivity. Whereas it is preferred that the host cells do not have the target bioactivity at all, low background levels may be tolerable if the effect of transformation (i.e. introduction of the second activity) is clearly distinguishable.

The second activity may lead directly to the production of a species which is a component part of the bioactive material or a precursor thereof (e.g. a peptide or protein component). Alternatively it may lead to the production of a species having one or more enzyme activities (e.g. being a polyketide synthase or part thereof), which enzyme activities are involved in the production of a component part or precursor. Of course, the roles of the activity already possessed by the cell and the introduced activity may be reversed.

Typically, some (or all) of the genes encoding proteins responsible for the biosynthesis of one or more naturally produced substances are placed on a single vector. It is then possible to genetically manipulate the pathway and transfer it or manipulated derivatives to other systems. Transfer to a complementary host possessing genes encoding biosynthetic pathway enzymes that can act in combination with the introduced genetic material causes production of biologically active materials. Use of complementary host cells that do not otherwise produce materials that have such biological activity allows the transformed cells to be directly screened for production of this biologically active material. This methodology is applicable to the manipulation, analysis and screening of the biosynthesis of many types of natural products or other biosynthetic pathways to produce novel chemical species and particularly for the screening of diversity that can be introduced to a biosynthetic cluster by a variety of molecular biological methods. Novel methods of introducing such diversity to a biosynthetic pathway are also provided.

There is a need to develop technologies that can utilise the genetic understanding and methodologies that lead to novel structural analogues of natural products and can transfer molecular diversity produced by such methods into easily screenable formats that can utilise the screening programs and high-throughput apparatus that have been developed by various biotechnology or pharmaceutical companies. Realistically, this means production and analysis of biologically active compounds directly from a genetically engineered cell without significant further manipulation. This can be readily achieved by genetically engineering a biosynthetic pathway, introducing the engineered pathway into a cell that acts in concert with the introduced pathway enzymes, and directly screening the transformed cells for novel bioactivity.

This need to develop new methodologies to generate and screen for novel bioactivities is particularly acute in the area of natural product biosynthesis, particularly in areas where methods to generate novel molecules using genetic or molecular biological techniques are being developed, and particularly biosynthesis of nonribosomal peptides and polyketides. In these cases there is a need to develop reliable and specific ways of deploying individual modules in practice so that all, or a large fraction of hybrid PKS genes that are viable and produce the desired fully processed, and hence screenable active product directly in a transformed cell, without the need for further transformation. Various and many strategies have been described to produce these hybrid PKSs particularly utilising recombinant DNA technology and de novo biosynthesis as described in the background art. But usually compounds made using these techniques have been assayed using traditional fermentation and chemical/visual analysis; biological assay to assess compound activity is performed much later in the process. Hence this invention represents a novel improvement on these techniques and particularly a method of utilising these tremendous advances in the field in a practical fashion. It is not feasible to ferment and chemically screen entire libraries containing hundreds of thousands of strains containing manipulated (randomly or otherwise) PKS or other biosynthetic pathways. A system designed for the direct production of active product enables pathways generated in a true combinatorial fashion to be analysed in a facile and rapid manner. There is a particular need to develop methods that allow direct screening of the engineered products without requiring a further step of biotransformation to active species. Many polyketides, for example, are not biologically active until modified by processing enzymes. It is particularly noteworthy that many of the advances in understanding of the mechanism of polyketide biosynthesis or development of novel polyketide structures through what has been termed ‘combinatorial biosynthesis’ has been performed utilising only the inactive macrolide core, effectively precluding their screening for activity without further purification and biotransformation or alternatively by fermenting transformants and producing a crude extract for bioassay and LCMS analysis (Tang L. and McDaniel R. (2001) 97, 1-9). A particular difference from these methods is that the present invention allows the direct in vivo modification of the PKS products to give active compounds, thus allowing direct screening of cells. Ease of screening is a vital characteristic of a usable combinatorial library. While possible, it is less feasible to screen large numbers of compounds from a strain library if they have to be biotransformed to give bioactivity after production. Direct activity screening allows newly produced strains to be analysed in standard formats utilising the high throughput methods that have already been developed. Biological screens can also be more sensitive than traditional chemical analytical methods.

In addition, there are other benefits of the approach. For example, there is a particular need to develop methods of generating and expressing PKS/hybrid PKSs in a manner that does not require gene replacement for every desired change since this is or can be laborious and time consuming. Directly screening transformants for activity provides a method of further verifying the genetic construct in addition to the marker used to select the transformants. It allows rapid detection of genetic constructs that have accumulated mutations or undergone recombination or deletion, a particular problem when using highly repetitive DNA as encountered in PKS or NRPS gene clusters. Screening transformants directly has the benefit of allowing better producing cells or colonies to be screened from poorer producers before fermenting to isolate the bioactive product. This is of particular utility in, for example, the actinomycete field, since it is generally accepted that many methods of transformation (particularly protoplasting) can cause additional genetic disruption and consequent decrease or indeed increase in product yield. Hence, a method that allows a rapid biological screen of production levels from many engineered transformants is of great utility since it can help screen out problematic strains.

The present invention differs from the ‘superhost’ type technologies in which entire or large portions of DNA are transferred to an alternative, often genetically cleaned, strain for screening by use of a complementary host-vector pair system. In our system, the vector generally contains portions of the pathway or manipulated derivatives, and the strain is chosen specifically to be able to activate the chemical products of the biosynthetic pathway to produce a screenable bioactive compound. Furthermore, it is envisaged that in particularly preferred embodiments of the invention much higher levels of information about the nature of the introduced DNA will be known, i.e. not just random genomic DNA or a biased genomic library. For example, in many cases the introduced pathway genetic material will have been engineered behind a specific promoter chosen because it is active in the host cell. It could have been manipulated in some way, in a directed manner, e.g. by substitution of a domain or site directed mutagenesis, or in a random manner, e.g. by introduction of random mutations using error prone PCR, transposons etc but not limited to these techniques. It is likely in many cases that much of the DNA sequence will be known. Furthermore, the requirement for the introduced material to act in concert with biosynthetic enzymes contained in the host cell means a greater level of knowledge about the likely function of the introduced DNA. Also, while not a requirement of the invention, in some embodiments the DNA is introduced by chromosomal integration. It is well known to those skilled in the art that considerable care must be taken in experimental design when introducing DNA by single cross to avoid problems with the transcript or downstream polar effects. There is also a requirement to provide areas of homology on the incoming DNA. Conversely, in the present invention host cells are specifically chosen to play a role in the production of the active compound; it is envisaged that a far wider range of host cells will be utilised than are currently used by researchers utilising ‘superhost’ technologies. A requirement of the invention is that the host cells contribute at least one enzyme activity to the screened bioactivity. This requirement not only helps to overcome size limitations of cloning vectors or situations where biosynthetic pathways are split over two genetic loci but greatly increases the potential for diversity using this invention since it is intended that manipulated pathways can be introduced into a range of cells each possessing a different modifying enzyme or enzymes. Hence, diverse libraries containing a portion of a biosynthetic pathway might be given further diversity by transferring the libraries into a range of cells, each set of which can be directly screened for activity.

Accordingly, in a first aspect the present invention provides a method for producing a biologically active product by transferring nucleic acid into a cell and directly screening that cell for bioactivity, the method comprising:
a) Selection or creation of a host cell lacking the target bioactivity but possessing some enzymic activities that will contribute to the biosynthesis of molecules that will exhibit that activity.
b) Introduction of DNA encoding part of a biosynthetic pathway into this cell.
c) Growth and screening of the cell for the target bioactivity.
d) Optionally, isolation of the target bioactivity from productive or interesting cells, e.g. for chemical analysis

In a further aspect the present invention provides host cells transformed with nucleic acids that can be screened for bioactivity, wherein, the biosynthetic pathway components encoded on the transferred nucleic acid produce one part of a chemical structure with bioactivity and biosynthetic pathway components encoded within the host cell produce alternative parts of the chemical structure. Host cells are selected for the presence of a particular enzymic activity contributing to biosynthesis of the bioactive molecule. Preferred host cells include prokaryotic microorganisms such as Eubacteria and Archaea bacteria, lower eukaryotic microorganisms such as fungi, algae and protozoa, and higher eukaryotic organisms such as plants and animals. Host cells can be derived from uni- or multicellular organisms. Any cell type can be used including cells that have been cultured in vivo or genetically engineered, with the proviso that the cell must possess an enzymic activity that contributes to the biosynthesis of the desired bioactivity and must lack the target bioactivity. A particularly preferred cell is a prokaryote or a fungal cell or a mammalian cell. A preferred host cell is a prokaryote, more preferably host cell strains such as Pseudomonas, myxobacteria, E. coli. Even more preferably the host cell is an actinomycete, even more preferably strains such as Saccharopolyspora erythraea, Streptomyces coelicolor, Streptomyces avermitilis, Streptomyces griseofuscus, Streptomyces cinnamonensis, Streptomyces fradiae, Streptomyces eurythermus, Streptomyces longisporoflavus, Streptomyces hygroscopicus, Saccharopolyspora spinosa, Micromonospora griseorubida, Streptomyces lasaliensis, Streptomyces venezuelae, Streptomyces antibioticus, Streptomyces lividans, Streptomyces rimosus, Streptomyces albus, Streptomyces rochei, Actinoplanes Sp., Amycolatopsis mediterranei, Nocardia Sp. and Streptomyces tsukubaensis. An alternative series of preferred host cells are Eukarote, a particularly preferred host is a mammalian, plant, yeast, or fungal cell. A still more particularly preferred eukaryotic host cell is a fungal strain, more preferably strains such as A. terreus and, A. nidulans.

In considering the enzymic activity carried by the host cells it is not intended to include enzymic activities that typically lead to production of precursor molecules such as malonyl-CoA or amino acids, components typically made by enzymes of primary or central metabolism. We are concerned with activities that typically contribute to secondary metabolic pathways, e.g. glycosyation, oxidation, deoxysugar biosynthesis etc, activities that are often, but not necessarily, clustered with other components of that pathway. This would include activities of polyketide unusual starter unit biosynthesis or unusual extender unit (i.e methoxy malonyl CoA synthesis).

Moreover the process of host cell selection further comprises the optional step of deleting or inactivating or adding or manipulating genes in the host cell. These genes might be involved in the production of some bioactivity, or be involved in the processing of the desired bioactivity.

In a further aspect, the present invention provides a process for producing a bioactivity, which process comprises culturing the transformed host cells identified by the process defined above and isolating the product thus produced. A preferred bioactivity is an activity that can be screened biologically. Particularly preferred bioactivities include antibacterial, antifungal, anticancer, antiviral, motilide, insecticidal, anthelmintic, herbicidal, anticoccidal, anticoagulant, anti-inflammatory, antiprotozoal, antiplatelet, anti-hypertensive, antiproliferative, proliferative, neuroregenerative, hair growth promoting, anti-fibrotic, antimalarial, antiplasmodial, antiangiogenesis, anticholesterol, cytotoxic, protein inhibition, protein synthesis inhibition and immunosuppressant among others

In further aspects the present invention provides novel bioactive products as obtainable by any of the processes disclosed herein. These novel bioactive molecules include any biologically active molecule that can be screened biologically. A particularly preferred biologically active molecule is a small molecule or a macromolecule. A preferred small molecule is a natural product, even more preferably a secondary metabolite. Particularly preferred secondary metabolites include polyketides, nonribosomal peptides, mixed polyketide-nonribosomal peptides, fatty acids, terpenes, alkaloids, aminoglycosides, shikimic acid derivatives, flavonoids, coumarins, taxol, steroids and other hormones. Preferred polyketides are those produced by type I or type II or fungal polyketide synthases. Examples of polyketides produced by type I PKS include 14-membered macrolides such as erythromycin or pikromycin, 16-membered macrolides such as tylosin, or macrolides such as avermectin, rapamycin, rifamycin, soraphen, spinosyn, FK506, FK520, borrelidin, geldanamycin, ansamitocin, maytansine, herbimycin, or epothilone polyenes such as amphotericin, nystatin or pimaricin, polyethers such as monensin or salinomycin, Examples of polyketides produced by type II PKS include daunorubicin, oxytetracycline or mitomycin. Examples of polyketides produced by fungal PKS include lovastatin. Preferred macromolecules include proteins, enzymes, peptides, polysaccharides, polyglycosides, nucleic acids. To emphasise, a requirement of the invention is that both the host cell and the transforming DNA contribute to the final biological activity. In the case of macromolecules this may be achieved by for example glycosylation, methylation, phosphorylation, acetylation, or addition of a subunit. These activities could lie in the cell, and the macromolecule be introduced by expression of the incoming DNA, or vice versa.

In a further aspect the invention provides vector systems suitable for performing the invention. Preferred host vector systems and methods for introducing the DNA into the cells are known to those in the art. These include plasmid vectors, BACs and cosmids among others. Vector systems must be compatible with the host. The use of shuttle vectors that can be maintained in multiple organisms may be beneficial. When more than one gene is required to be expressed in said host cell, many ways will readily occur to the person skilled in the art as to how to achieve this goal in such a way that leads to coordinated expression of all the required gene products. Preferred nucleic acids that are inserted in these vectors are nucleic acids encoding enzymes participating in biosynthetic pathways such as genes encoding polyketide synthases, non-ribosomal peptide synthases, glycosyltransferases, methyltransferases, cytochrome P450, deoxysugar biosynthetic pathways, terpene cyclases and shikimate pathway enzymes.

In a further aspect the present invention provides a method for producing a library and screening it for one or more desired bioactivities, the method comprising:
a) Selection or creation of a host cell lacking the or each target bioactivity but possessing some enzymic activity that will contribute to the biosynthesis of that activity.
b) Generation of a library of nucleic acids
c) Introduction of nucleic acids into the cells, to produce a plurality of cells each containing a different nucleic acid.
d) Growth and screening of the cells for the or each target bioactivity.
e) Optionally, culturing of the cells and isolation of bioactive product from cells producing a target bioactivity.

In preferred embodiments the library produces at least 2 bioactive products, more preferably at least 10 bioactive products, more preferably at least 50 bioactive products and still more preferably at least 100 bioactive products, generally from respective different cells or cell populations. Cells in the library may also differ in quantity, strength or type of bioactivity.

A process may further include isolating a host cell producing a desired bioactivity and treating it further (e.g. culturing the cells and isolating the product produced) so that the product can be made in bulk.

In a particularly preferred type of embodiment the present invention relates to processes and materials (including enzyme systems, nucleic acids, host cells, vectors and cultures) for preparing and detecting ketides, particularly novel polyketides, such as 12-, 14-16- or larger-membered ring macrolides, polyethers or polyenes by recombinant synthesis and to the novel polyketides so produced. Polyketide biosynthetic cluster genes or portions of them, which may be derived from different polyketide biosynthetic gene clusters, are manipulated to allow the production of specific novel polyketides, such as 12-, 14- and 16-membered macrolides, usually of predicted structure and expressed in a strain capable of making or modifying a polyketide chain to give biological activity.

In one preferred type of embodiment the invention is concerned with the use of (a) a vector containing PKS genes of a cluster or derivatives and (b) a complementary but specifically prepared or selected host strain that possesses biosynthetic pathway enzymes which, when expressed together with the vector encoded biosynthetic pathway genes, will give a desired bioactivity. However, a number of other potential combinations will readily occur to a person skilled in the art. For example a vector which contains enzymes that generally act after polyketide chain formation, for example one or more of glycosyltransferases, pathways to TDP-sugars, cytochrome P450s, and methylases, may be used with a host strain which expresses the PKS that produces the polyketide on which they will act. Alternatively, in the converse case the strain may be capable of making a polyketide synthase or manipulated derivative and the vector contains the biosynthetic pathway enzymes that generally act after polyketide backbone formation. Alternatively, a strain might have part of a PKS and the transforming vector another part of the PKS. This might take a number of forms; the strain might be deleted in one or more PKS open reading frames and the vector used to express these reading frames (or derivatives). The strain might possess a vestigial part, even a non-complete portion of a PKS and the vector used to express the rest of the PKS. Alternatively the strain might be unmodified or unrelated to the transforming PKS. The need for precise design of the strain and introduced genetic material will often depend on the particular cell type, for example to maintain reading frame, and maintain transcription and avoid polar effect, such considerations will be known to those skilled in the art.

In certain embodiments of the invention the strain may possess an area of homology to a region on the vector to allow chromosomal integration. For example this area of homology might be a small part of the PKS, e.g. the thioesterase domain. Alternatively, the inserted DNA might be directed to a neutral site on the chromosome or an attachment site. Alternatively, if the strain permits, the vector may be self-replicating. When a PKS-encoding vector is transformed into this strain and the PKS is expressed, polyketide products can be fully processed to give biologically active materials allowing transformants to be directly screened for novel activities or for product yields. Alternatively, in the opposite case, transformation of the PKS-containing strain with a vector capable of activating the polyketide produced by that PKS-containing cell (for example by glycosylation or hydroxylation) leads to production of biologically active material.

A particular feature of this strain is that it does not possess the biological activity until transformed by the biosynthetic pathway. It is well known in the art that a number of methods can be used to remove ‘unwanted biological activity’ prior to use of the strain including random mutagenesis using, for example, ultraviolet light or chemical mutagens and screening for organisms that no longer display the activity. In some cases biosynthetic pathways leading to bioactive products can be more precisely deleted (Rowe et al. (1998) Gene, 216, 215-223 or McDaniel et al. (1993) Science, 262, 1546) this may be necessary and a benefit if the aim is to recombine a manipulated pathway into the original host since it may remove the need to perform the experiment by gene replacement. Media development techniques can be used to find conditions that close down the pathways that lead to production of the competing activities. In all cases care must be taken not to influence the ability of the strain either to make or activate the products of the introduced pathway enzymes. Although it is desirable that the strain would not display the target bioactivity at all, low background levels are acceptable as long as the expressed bioactivity can be readily distinguished against the background. It may be necessary to determine conditions that are compatible with the transformation protocol and bioactivity production, for example use of assays that are not affected by the antibiotics used to select for transformants, or use of additional simple stages to remove the transformants from the selective media. It is however within the scope of this invention to perform replica plating, picking to 96 well plates or similar methods for growth and expression of the bioactivity. This may have the added advantage of removing the antibiotic used to select for the transformants, which may in some cases influence the bioactivity screen. Such steps may also aid considerably the use of the invention described herein in existing high throughput screening systems.

Additionally, the present invention teaches that it is possible to take an entire PKS or portions thereof, on a single vector, and manipulate the PKS by conventional methodology now well established by researchers in the field utilising targeted methods. For example, convenient restriction sites may be used to swap selected domains, or random methods may be used, for example, directed evolution. Subsequently the entire PKS is used to transform a strain specifically chosen/designed to modify the chemical products of the manipulated PKS to give active products. Usually the chosen strain will not possess a particular biological activity that the polyketide may possess. This allows transformants (clones initially selected by antibiotic resistance contained on the vector) to be directly screened for bioactivity. For example, recombinant colonies with a specific bioactivity can be selected on the plate (or a replica) for novel antimicrobial activity by overlaying the plate with a susceptible organism. It is not intended to limit the invention to antimicrobial screens in which the transformed cells or replicas can be directly overlayed with a test organism. It is well known to those skilled in the art that extracts could be taken from the transformed colonies growing on a transformation plate or similar and used for bioassay—this may particularly apply to protein based assay screens.

Other uses of the invention can be envisaged. For example, higher yielding colonies may be directly selected after transformation; this is particularly useful because many conventional methods of actinomycete transformation (particularly those utilising protoplast formation) result in significant drops in yield probably due to secondary deleterious mutations. These mutations can also result in enhanced production levels, and higher producing strains can also be directly detected by the disclosed methodology which can be of particular benefit to industrial scale-up processes.

In a particularly preferred embodiment, the vector containing the manipulated PKS will be returned to the strain from which the PKS originated, after the strain itself has been manipulated, e.g. by deletion of most of the open reading frames responsible for production of the original polyketide and the genes or modified versions placed on the vector to be modified. However, this is not necessary but helps ensure enzymic activities (i.e. in this case post PKS enzymes) possessed by the strain are likely to have the catalytic ability to act on the chemical products of the proteins carried by the vector. In addition the strain is more likely to possess a suitable substrate supply for building the desired molecules. However this invention is not limited to use of strains related to the vector encoded PKS/genetically manipulated PKS. For example, the PKS might be inserted into a strain specifically chosen to possess a particular biotransformation ability, converting the non-active form of the polyketide to an active, and hence directly screenable compound. In considering the enzymic activity contributed by the strain, it is not intended to limit the invention to biosynthetic activities that activate the product to give bioactivity, e.g. glycosylation of erythronolide to give an active erythromycin. The opposite case could apply: the biosynthetic activity carried by the strain could be the PKS itself and the genetic material introduced be a glycosyl transferase or an alternative or modified sugar pathway. A key aspect of the invention is that the resulting strain is directly screened for a bioactivity after transformation of the strain. To emphasise, while in many of the embodiments described we use the example of introducing a PKS into a strain and screening for the activity conferred on the polyketide by the action of biosynthetic enzymes within the cell, in each case the opposite could apply, ie. introducing biosynthetic enzymes to a strain possessing a PKS and screening for bioactivity conferred on the cell by that action.

The invention allows preparation of a modified PKS or biosynthetic pathway, or a portion of a PKS or biosynthetic pathway on a single vector under the same promoter region. This is particularly useful if the entire PKS is usually transcribed from the same promoter. However, it will be well known by those skilled in the art that the same techniques can be used for PKSs that are not expressed from the same transcript although this may be more complicated. Alternatively the genes can be engineered to be under the control of a single promoter. Possible methods will be known to those skilled in the art, Gaisser S. et al. (2000) Mol. Microbiol, 36, 391-401 illustrates an alternative method by which genes may be placed under the control of one promoter. A particular advantage of the present invention is that all the required changes to prepare the modified/heterologous PKS can be made in E. coli prior to transfer to the host strain and subsequent screen. An advantage of the invention is that because transformants are screened for bioactivity this is an easy check that an engineered plasmid construct (or other vector) is correct and functional. This is a particular advantage in systems where the plasmid construct is extremely large or inherently unstable to recombination, where conventional methods of screening for plasmid fidelity are difficult or inconclusive. This situation particularly applies to PKS- or NRPS-containing constructs where the highly repetitive nature of the modules can lead to deletion or rearrangement. A further advantage of the present invention is that it effectively avoids the need to conduct all the gene replacement experiments in pathways by double crossover, a process that can be time consuming and laborious, indeed in some strains, impossible, particularly in PKS regions. Changes may be made in E. coli using the standard molecular biological methods used in the art and the final vector transferred to the host and screened for bioactivity. In designing/selecting the transfer vector a consideration is that a recombinant strain containing a vector integrated into the chromosome is believed by many in the pharmaceutical industry to possess significant advantages over a strain containing a self-replicating vector, particularly in large-scale fermentations. However, it is not intended to limit the invention only to systems that involve chromosomal integration. For research purposes there can be considerable benefit in speed and ease of use if the introduced DNA can be introduced on a self-replicating vector, if one is available for that host strain. Alternatively the DNA can be introduced by conjugation, often directed to an attachment site, of which there are a range of choices. This will be well known to one skilled in the art. Use of a self-replicating vector often avoids the need to consider the effect of the chromosomal integration due to polar effects or changes to the transcript. Indeed, since in many embodiments of the present invention a vector containing a manipulated cluster is returned to a derivative of the originator strain there are commercially relevant benefits. Often the pharmaceutical industry has extensive experience in fermenting a particular strain, both for optimising product yield and limiting byproducts, shunt metabolites etc. While it is envisaged that original experiments may be performed in a laboratory strain to take advantage of ease of transformation or other preferred characteristics, it is intended that in many cases the large vector system can be subsequently applied to a derivative of an industrial production strain with minimal additional effort. Potential methods of introducing DNA into the host will be host dependent and be known to those skilled in the art

In another aspect the invention provides a method of making changes to a polyketide chain by manipulating the genes responsible for its production. The genes are placed on a single vector under one or more promoters, usually but not necessarily on the same transcript. Changes are made to the desired regions of the PKS genes in a convenient organism, e.g. E. coli and the final vector is transformed into the host strain by single integration. The host is then screened for activity. This method is particularly useful for making changes in PKS modules lying away from the promoter regions/terminal regions, changes that usually have to be made laboriously by gene replacement, rather than by single integration. Changes to a PKS for example, could include: loading module swaps, extender module swaps, domain swaps (Acyl transferase, reductive domains), multi-domain swaps, partial domain swaps, deletions or insertions, truncations or extensions, exchanges, including site directed mutagenesis.

In another aspect this invention provides host strains specifically deleted of whole open reading frames of a PKS. Usually it may be beneficial not to delete the entire PKS as the remaining portion may act as an area of homology. A number of scenarios can be envisaged, i) deletion of whole open reading frame(s) and ii) deletion of less than an entire open reading frame. These strains are unable to produce the specific polyketide, but retain post PKS processing activities and thus are able to process molecules that are produced when the strains are transformed with constructs designed to replace the deleted portion of the PKS. Deletion of large portions of the PKS prevents or reduces the risk of unwanted recombination between the homologous regions on the vector and chromosome, thus directing the desired recombination event. The deletion of the PKS may also have the effect of removing a biological activity of the strain, allowing the organism to be screened directly. These strains may be further modified after deletion of open reading frame(s) to enhance or confer, for example, particular desired post PKS activities that will act to give the polyketide screenable activity.

In another aspect the invention provides a simple method of analysing the effect of moving a PKS from one host to another to take advantage of alternative post PKS processing routes, such as operation of different glycosyl transferases or cytochrome P450s to produce novel glycosylated or oxygenated derivatives. In addition this methodology may be of importance if the structure of a polyketide produced by a manipulated PKS differs significantly from that normally utilised by the post PKS processing enzymes.

In another aspect the invention provides a method of rapidly screening randomly produced PKS enzymes (produced for example by combinatorial biosynthesis or directed evolution/gene shuffling but not limited to these techniques) for efficacy/improved catalytic function/specificity or product yield. Random changes are made to the PKS containing vector to generate a library of different PKS genes, perhaps differing by only one nucleotide or possessing entirely different modules, domains or open reading frames, or insertions or deletions etc. This library is transformed into the host strain, vector-containing hosts are selected by resistance marker, and transformants are directly screened for bioactivity. Screening for bioactivity directly after transformation into a strain that can activate the molecules so produced screens for the productive changes. Novel methods of producing such a library are outlined below. Transformation of the library into a range of different host strains possessing different modifying enzymes yields differently processed molecules that possess different activities.

In another aspect the invention provides a novel method of generating a library of bioactivities. This method is applicable to polyketides, non-ribosomal peptides and many other secondary metabolites or natural products, particularly when the protein/domain order is similar within that class. The method involves generation of a series of strains specifically deleted of all or part of an open reading frame of a PKS cluster except the N-terminus and C-terminus of a particular open reading frame, or alternatively deleted in all/part of a domain. Specific complementation of the missing section by DNA containing one or a multiplicity of modules or domains, by cloning an equivalent section derived from a heterologous source, between these domains reforms the open reading frame consequently returning the activity. The activity in the strain can be screened directly. Multiple fragments or a library of fragments can be screened to determine the most effective. The source of these fragments could be from a range of known clusters or from unknown sources e.g. environmental samples. It is known to those skilled in the art that the ability to rapidly and easily test a range of fragments confers a considerable advantage to a researcher since a useful fragment may act less efficiently in another context. In an extension to this aspect, as another way of generating a library, random fragments of unknown PKS can be cloned by amplification using degenerate primers made using regions of strong homology and incorporating suitable restriction sites. By placing such degenerate fragments into the system, novel polyketides might be produced and screened directly. In the context of PKS the amplified product might be a domain, a module or multiple modules, and in fact it does not matter if the replaced fragment is not a single reading frame if the gene organisation is maintained. The vector-host strain combination methodology of the present invention enables a researcher to rapidly screen the successful transformants by biological activity and hence is a tool that allows a researcher to determine the most efficient swap. It is well known in the art that determination of the most appropriate swap is an inexact science; in many cases different replacements give different yields. Direct screening of the biological activity means the present invention may be used to screen multiple (in principle thousands) of swaps, directly from transformed colonies, judging the success of swaps by screening the transformed colonies for both novel activity/product yield.

This invention has a wide potential utility and is not restricted to polyketides or indeed to microbial natural products. The method is applicable to any multi-step biosynthetic pathway that produces a product that can be screened biologically. The genes encoding the biosynthetic machinery responsible for the production of many bioactive metabolites are rapidly being discovered, a task made easier by the fact that many are found clustered together. However, advances in high-throughput DNA sequencing methods, including the sequencing of entire genomes or gene discovery methods such as described by Santi D. V., et al (2000) Gene, 247, 97-102 means even biosynthetic machinery that is not clustered can be readily identified. Once identified it is possible, as we have illustrated for a PKS, to place these genes on a vector, perhaps into a cassette utilising stronger promoters to enhance, or better control expression levels. However, even when the biosynthetic machinery of interest is not clustered there are methods available to place the genes onto a single vector. This vector can be transformed into host cells chosen because i) they can participate in the biosynthesis of the desired novel bioactive products produced together with the vector encoded proteins (perhaps the host cells possess the remainder of the cluster that is not clustered with the identified genes) and ii) because they do not possess a particular bioactivity (or not to any substantial extent) allowing transformed cells to be screened directly. As we illustrated for a PKS the host cells may be first manipulated to remove a particular biosynthetic machinery. This may also remove a biological activity. Alternatively the cells may be manipulated to contain a particular desired pathway enzyme. Transformation of these cells with a vector containing the original genes (or manipulated derivatives thereof) restores the biological activity, allowing the cells to be screened for productive events and bioactivity.

It is not intended to limit the scope of the invention to ‘traditional’ natural products. The principles behind the invention apply to far wider classes of biosynthetic products. These might include for example, protein libraries. We can envisage a method in which expression of desired protein is placed under the control of a vector that is subsequently transformed into a host strain containing enzymes responsible for glycosylation or acetylation of the introduced encoded protein to produce a bioactive protein that can be screened directly from the transformant. Alternatively the host strain might be able to synthesise a second subunit of a protein, expression of the other subunit on a plasmid would give the screenable bioactive product.

Embodiments of the present invention will now be described by way of example and with out limitation with reference to the accompanying figures, in which:

FIG. 1 is a diagram of the functioning of 6-deoxyerythronolide synthase (DEBS) a modular PKS producing 6-deoxyerythronolide B, a precursor of erythromycin A.

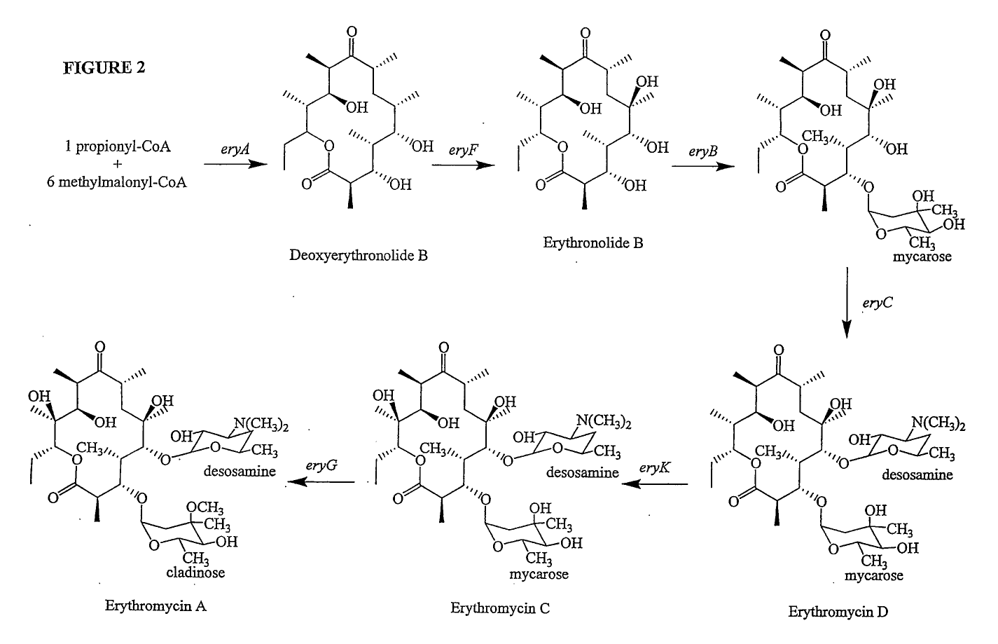

FIG. 2 shows the enzymatic steps that convert 6-deoxyerythronolide B into erythromycin A.

### EXAMPLE 1

Construction of Plasmid pHP020

Plasmid pHP020 is a pCJR24-based plasmid containing DEBS1, DEBS2 and DEBS3 under the control of the actI promoter. The AT domain of DEBS2 (module 4) has been exchanged with the AT domain of rapamycin module 2. Plasmid pHP020 was constructed by several intermediate plasmids as follows. Plasmid pIB023 containing DEBS1, DEBS2 and DEBS3 was digested with MscI and a 4.7 kb fragment isolated by gel electrophoresis and purified from the gel (MscI site not dcm sensitive). [Plasmid pIB023 was produced by plasmid rescue of EcoRI fragments isolated from genomic DNA of strain Sacch. erythraea CJR65 that contains pCJR65 (DEBS1TE in pCJR24, Rowe C. J et al. (1998) Gene 216 215-223).

Plasmid pHP010 is a derivative of this plasmid containing 1 kb sequence from downstream of the TE domain (cloned EcoRI-XbaI) to provide extra homology for transformation into Sacch. erythraea JC2]. The MscI fragment was ligated to plasmid pUC19 that had been linearised with SmaI and treated with alkaline phosphatase. After transformation into E. coli DH10B and identification of the plasmid pHP001 a 4.7 kb fragment was excised with EcoRI and XbaI and ligated with a 3.3 kb fragment formed by digestion of pCJR24 with EcoRI and SpeI. The ligation mixture was used to transform electrocompetent E. coli DH10B cells and individual clones checked for the desired plasmid pHP005.

Two oligonucleotide primers were used to amplify DNA from the Sac. erythraea eryAII gene extending from nucleotide 27227 to 28493. 5′-TTTTTCTGCAGCGCCCTGGCCAGGGAAGACCAGGACCG-3′ and 5′-TTTTTAAGCTTCCTGCGAGGCACCGACACCGGCG-3′ using pHP001 as a template. The design of the primers incorporated an MscI site at the nucleotide 27231 and a PstI site located just before the MscI site and the second a HindIII site priming across a SfII site. The 1276 bp PCR product was digested with PstI and HindIII and ligated into pUC19 that had been digested with the same enzymes. The ligation mixture was used to transform electrocompetent E. coli DH10 cells and individual clones checked for the presence of the desired plasmid pHP004. Plasmid pHP004 was identified by restriction pattern and confirmed by sequence analysis.

Two oligonucleotide primers were used to amplify DNA from the Sac. erythraea eryAII gene extending from nucleotide 26269 to 25751. 5′-TTTTTGAATTCCGTCCTCCGGCGGCCACTGCTCGG-3′ and 5′-TTTTTCTGCAGCCTAGGGGGACGGCCGGCCGAGCTGCCCACC-3′. The design of the primers incorporated an AvrII site at nucleotide 26273 and a PstI site located just before an AvrII site and the second an EcoRI site added to the nucleotide sequence at 25749. The 545 bp PCR product was digested with PstI and EcoRI and ligated into pUC19 that had been digested with the same enzymes. The ligation mixture was used to transform electrocompetent E. coli DH10B cells and individual clones checked for the presence of the desired plasmid pHP003. Plasmid pHP003 was identified by restriction analysis and sequence analysis.

Plasmid pHP003 was linearised with PstI and HindIII and purified by gel electrophoresis. The ˜3 kb fragment was treated with alkaline phosphatase and ligated to a ˜1.2 kb fragment of pHP004 that had been digested with PstI and HindIII and gel purified. The ligation mixture was used to transform electrocompetent E. coli DH10B cells and individual clones checked for the presence of the desired plasmid pHP007. Plasmid pHP007 was identified by restriction pattern. Plasmid pHP007 was digested with SfiI and a 2.2 kb fragment purified by gel electrophoresis and purified from the gel. This fragment was used to replace a SfiI fragment from pHP005 and resulting vector designated as pHP012, a replacement cassette without the presence of AT4. Alternative ATs can be introduced at the unique MscI and AvrII sites introduced during the engineering of this plasmid.

Rapamycin AT2 that specifies a malonate extender unit was used to replace AT4 from the Sac. erythraea eryAII gene. Plasmid pCJR26 was the source of rapamycin AT2 (Rowe C. J et al. (1998) Gene 216 215-223). Demethylated DNA was produced by transforming E. coli strain ET12567 with plasmid pCJR26. This DNA was digested with MscI and AvrII and a ˜0.9 kb fragment containing rapamycin AT2 was purified by gel electrophoresis and ligated to demethylated pHP012 that has previously been digested with the same enzymes, treated with alkaline phosphatase and purified by gel electrophoresis. The ligation mixture was used to transform electrocompetent E. coli DH10B cells and individual clones checked for the desired plasmid pHP014. Plasmid pHP014 was identified by restriction pattern.

Plasmid pHP014 was digested with SfiI (dcm non sensitive) and a 2.2 kb fragment isolated by gel electrophoresis and purified from the gel. This fragment was ligated to pHP010 that has been previously extensively digested with SfiI, treated with alkaline phosphatase and purified by phenol extraction and ethanol precipitation. The ligation mixture was used to transform electrocompetent E. coli DH10B cells and individual clones checked for the presence of the desired plasmid pHP020 by restriction analysis.

### EXAMPLE 2

Construction of Sacch. erythraea NRRL2338 JC2 (pHP020) and Screening it for Production of Novel Bioactive Erythromycins

Plasmid pHP020 was used to transform Sacch. erythraea NRRL2338 JC2 protoplasts using standard techniques. Sacch. erythraea NRRL2338 JC2 has been precisely deleted of the entire DEBS1+2+3 leaving the TE as a homology region for integration of PKS plasmids (Rowe C. J et al. (1998) Gene 216 215-223). The transformation mixture was plated onto R2T20 plates and recovered for 24 hours before overlaying with 40 μg/ml thiostrepton. Colonies were grown for approximately 7-10 days to allow sporulation before harvesting the spores and plating dilutions onto R2T20 agar containing 40 μg/ml thiostrepton and grown for 7 days at 30° C. Individual thiostrepton resistant colonies were patched in 1 cm2 areas onto a large (25×25 cm) square R2T20 agar plate (poured on a horizontal surface to ensure equal depth of agar) leaving reasonable space between each patch and grown for approximately 5 days to allow secondary metabolite production. At this stage small, plugs of agar were taken from each patch and placed at equally spaced intervals on a (25×25 cm) LB plate that had been overlayed with a culture of the indicator organism M. luteus. A patch of JC2 was used as a control. The LB plate was then placed at 4° C. for 4 hours to allow diffusion of natural products from plug to the LB agar before moving the plate 37° C. to allow growth of the screening organism. Individual plugs were screened by size of zone of inhibition, allowing screening of non-producers vs producer organisms but additionally screening for better producing colonies.

To further verify the introduction of the correct construct producer-clones were tested for the presence of pHP020 integrated into the TE region by Southern blot hybridisation of their genomic DNA with DIG labelled DNA containing ˜1 kb MscI/AvrII fragment of rapamycin AT2. All the producer-clones tested appeared to contain a correctly integrated copy of pHP020, as could be predicted by the formation of bioactive products. To verify the exact chemical nature of the active product identified by the screening method two tests were made. Firstly, utilising the remainder of the replica plated colony used to produce the plug for the screening organism. A second plug (˜0.5 cm²) was taken and extracted with 2×1 ml ethyl acetate that had been adjusted to ˜pH9 (ammonia). The solvent from these extracts was removed by evaporation and the residue analysed by HPLC/MS. A peak was observed with molecular mass m/z [M+H]+=690 for 6-desmethyl erythromycin D. Secondly, S. erythraea NRRL 2338 (pHP020) was used to inoculate 5 ml TSB containing 5 μg/ml thiostrepton. After three days growth 1.5 ml of this culture was used to inoculate 30 ml of EryP medium containing 5 μg/ml thiostrepton in a 250 ml flask. The flask was incubated at 30° C., 250 rpm for 6 days. At this time the supernatant was adjusted to pH 9 with ammonia and extracted twice with an equal volume of ethyl acetate. The solvent was removed by evaporation and the residue analysed by HPLC/MS. A peak was observed with molecular mass m/z [M+H]+=690 for 6-desmethyl erythromycin D. Further analysis showed the presence of products due to incomplete post PKS processing, B, C and A forms, and compounds due to product breakdown. Quantitation of the peak corresponding to 6-desmethyl erythromycin D indicated the product was produced at approximately 1 mg/L under the flask conditions used.

### EXAMPLE 3

Production of Polyketides by Fermentation of Sacch. erythraea NRRL2338 JC2 (pHP020)

To further verify the chemical nature of the product identified by the plate screening procedure we grew the culture at large scale. A glycerol working stock of Sacch. erythraea JC2/pHP020 stored at −80° C. was used to inoculate two 250 ml flasks with springs containing 50 ml TSB with 5 μg/ml thiostrepton and grown at 30° C. and 240 rpm on a shaker with a 2 inch throw for 2 days. This seed culture was used to inoculate at 5% v/v three 2 L flasks with springs containing 300 ml TSB with 5 μg/ml. These cultures were grown at 30° C. and 240 rpm on a shaker with a 2 inch throw for a further 2 days to provide a seed culture for the fermentor. The seed culture was used to inoculate at 5% v/v 12 L EryP medium in a 20 L Applikon fermentor system. EryP medium (Pacey, M. S., et al. (1998) J. Antibiotics 51, 1029-1034).

| | | |
|---|---|---|
| Glucose | 50 | g/L |
| Nutrasoy flour | 30 | g/L |
| Ammonium sulphate | 3 | g/L |
| Sodium chloride | 5 | g/L |
| Calcium carbonate | 6 | g/L |
| PPG antifoam | 0.1 | g/L |

Adjust to pH 7.0 using NaOH. Sterilise at 123° C., 30 minutes.

The broth was incubated at 30° C. with an aeration rate of 6 L/min, stirring at 200 rpm increasing to a maximum of 350 rpm during the fermentation. The fermentation was continued for approximately 120 hours. At this time the presence of the desired compound, 6-desmethyl erythromycin D, was confirmed by adjusting the pH of a broth sample to pH 9.0 with ammonia and extracting with an equal volume of ethyl acetate. The solvent was removed by evaporation and the residue analysed by HPLC/MS. Cell material was removed by centrifugation to leave 10 L cleared supernatant.

### EXAMPLE 4

Purification and Characterisation of 6-desmethyl Erythromycin D

The supernatant (10 L) was adjusted to pH10 with sodium hydroxide and stirred with Amberlight XAD-16 resin (100 g) for 30 minutes at room temperature. The resin was isolated by filtration, washed with water (200 ml) and compounds eluted with methanol (3×200 ml). This step was repeated twice and resulting methanol extracts combined and the sovent removed in vacuo to leave a aqueous residue (˜50 ml) The extract was diluted with water and extracted with ethyl acetate (3×100 ml). The extracts were combined and solvent removed in vacuo to give an oil (6.9 g). The oil was partitioned between acetic acid/acetate (1:1, 100 ml, pH5) and ethyl acetate (100 ml). The pH of the aqueous phase was adjusted to pH9.5 with aqueous ammonia and back extracted with ethyl acetate (100 ml). The solvent was removed to give a concentrated extract (˜0.5 g). Significant loss was tolerated at this stage to ensure purity. The extract was further purified by reverse phase (C18) preparative HPLC utilising a Gilson 315 system and a 21 mm×250 mm 5 μm Hypersil BDS C18 column. A linear gradient elution of 25%-75% acetonitrile/20 mM ammonium acetate over 19 minutes at a flow rate of 21 ml/minute was used. Fractions were analysed by LCMS to identify those containing 6-desmethyl erythromycin D, pooled and solvent removed in vacuo. Other 6-desmethyl erythromycins were identified in other fractions. The 6-desmethyl erythromycin D containing residue was bound to an isoelute ENV+cartridge (200 mg), washed with water to remove buffer and eluted with methanol (6 ml) to yield 6 desmethyl erythromycin D (4.2 mg). The structure of 6-desmethyl erythromycin D was confirmed by NMR spectroscopy (see Table I).

TABLE 1
¹H and ¹³C NMR data for 6-desmethyl erythromycin D

| Position | δ_{H} | Multiplicity | Coupling | δ_{C} |
|---|---|---|---|---|
| 1 | | | | 176.4 |
| 2 | 2.67 | qd | 7.0, 5.1 | 44.8 |
| 3 | 4.43 | dd | 5.1, 1.5 | 82.5 |
| 4 | 2.02 | m | | 40.5 |
| 5 | 3.59 | m | | 81.8 |
| 6 | 4.26 | m | | 74.4 |
| 7 | 1.92 | m | | 33.1 |
| | 1.84 | m | | |
| 8 | 2.64 | m | | 47.7 |
| 9 | | | | 217.5 |
| 10 | 3.03 | qd | 7.0, 1.7 | 41.5 |
| 11 | 3.91 | d | 9.6 | 70.0 |
| 12 | 1.75 | m | | 38.7 |
| 13 | 5.18 | ddd | 8.1, 6.6, | 75.8 |
| | | | 1.7 | |
| 14 | 1.80 | m | | 25.1 |
| | 1.50 | dqd | 14.1, 7.3, | |
| | | | 4.3 | |
| 15 | 0.89 | dd | 7.5, 7.5 | 10.1 |
| 16 | 1.21 | d | 7.0 | 13.6 |
| 17 | 1.10 | d | 7.0 | 9.7 |
| 18 | 1.18 | d | 7.0 | 17.9 |
| 19 | 1.00 | d | 6.8 | 8.5 |
| 20 | 0.91 | d | 7.0 | 9.1 |
| 1′ | 4.87 | d | 3.2 | 100.1 |
| 2′ | 1.79 | m | | 40.5 |
| | 2.26 | dd | 14.7, 1.1 | |
| 3′ | | | | 69.8 |
| 4′ | 2.98 | d | 9.6 | 76.3 |
| 5′ | 3.86 | dq | 9.6, 6.4 | 66.4 |
| 6′ | 1.33 | d | 6.4 | 17.8 |
| 7′ | 1.25 | s | | 25.4 |
| 1″ | 4.25 | d | 7.3 | 105.6 |
| 2″ | 3.27 | dd | 10.2, 7.0 | 70.2 |
| 3″ | 2.61 | m | | 65.4 |
| 4″ | 1.75 | m | | 29.1 |
| | 1.29 | m | | |
| 5″ | 3.54 | m | | 69.5 |
| 6″ | 1.23 | d | 6.2 | 21.2 |
| 7″ | 2.35 | s | | 40.3 |
| 6-OH | 3.09 | br. s | | |

### EXAMPLE 5

Construction of Plasmids pLS025 and pSGK047

Plasmid pLS025 is a pCJR24-based plasmid containing the DEBS1, DEBS2 and DEBS3 in which the loading domain of DEBS1 has been replaced by the loading domain of the avermectin biosynthetic cluster. Plasmid pSGK047 is a SCP2 based plasmid containing the same genes.

Plasmid pLS025 was constructed as follows. Plasmid pIG1 (Marsden et al. (1998) Science 279, 199-202) was digested with NdeI and XbaI and the ˜12 kb, PKS containing fragment isolated by gel electrophoresis and the fragment purified from the gel. This fragment was ligated into pCJR24 that been digested with the same enzymes and purified in the same manner. The ligation mixture was used to transform electrocompetent E. coli DH10B and individual clones checked for the presence of the desired plasmid pLS005. Plasmid pLS005 was identified by restriction pattern. Plasmid pLS005 was digested with ScaI and the ˜10 kb fragment isolated by gel electrophoresis and purified from the gel. Plasmid pHP010 was digested extensively with ScaI and dephosphorylated to ensure the fragments could not religate. At this stage all transfer of DNA was performed with wide bore tips to avoid shearing of DNA. Digested pHP010 was ligated with the fragment derived from pLS005. The ligation mixture was used to transform electrocompetent E. coli DH10B and individual clones checked for the presence of the desired plasmid pLS025. Plasmid pLS025 was identified and confirmed by restriction pattern, since one ScaI site is situated in the ampicillin resistance gene orientation of fragment is predefined.

Plasmid pSGK047 was constructed as follows. Plasmid pLS025 was digested with NdeI and XbaI and dephosphorylated to ensure the fragments could not religate. Plasmid pCJR29 (Rowe et al., (1998) Gene 216, 215) was digested with NdeI and XbaI to remove the polylinker, isolated by gel electrophoresis and purified from the gel. Digested pLS025 was ligated with this fragment. The ligation mixture was used to transform electrocompetent E. coli DH10B and individual clones checked for the presence of the desired plasmid pSGK047. Plasmid pSGK047 was identified and confirmed by restriction pattern.

### EXAMPLE 6

Construction of Sacch. erythraea NRRL2338 JC2 (pLS025) and its Use and Screening to Produce Novel Erythromycins

Plasmid pLS025 was used to transform Sacch. erythraea NRRL2338 JC2 protoplasts using standard techniques. The transformation mixture was plated onto R2T20 plates and recovered for 24 hours before overlaying with 40 μg/ml thiostrepton. Colonies were grown for approximately 7-10 days to allow sporulation before harvesting the spores and plating dilutions onto R2T20 agar containing 40 μg/ml thiostrepton and grown for 7 days at 30° C. This additional step avoids some of the false positive colonies observed when transforming S. erythraea but could be omitted as the activity screen will also screen out these colonies. Individual thiostrepton resistant colonies were patched in 1 cm2 areas onto a large (25×25 cm) square R2T20 agar plate and containing 10 μg/ml thiostrepton, leaving reasonable space between each patch and grown for approximately 5 days to allow secondary metabolite production. At this stage small, plugs of agar were taken from each patch and placed at equally spaced intervals on a (25×25 cm) LB plate that had been overlayed with a culture of a thiostrepton resistant strain of the indicator organism B. subtilis. A patch of JC2 was used as a control. The LB plate was then placed at 4° C. for 4 hours to allow diffusion of natural products from plug to the LB agar before moving the plate 37° C. to allow growth of the screening organism. Individual plugs were screened by size of zone of inhibition, allowing screening of non-producers vs producer organisms but additionally screening for better producing colonies.

To verify the exact chemical nature of the bioactive product the best producing colony (as determined by the bioactivity screen) of S. erythraea NRRL 2338 (pLS025) was used to inoculate 5 ml TSB containing 5 μg/ml thiostrepton. After three days growth 1.5 ml of this culture was used to inoculate 30 ml of EryP medium containing 5 μg/ml thiostrepton in a 250 ml flask. The flask was incubated at 30° C., 250 rpm for 7 days. At this time the supernatant was adjusted to pH 9.0 with ammonia and extracted twice with an equal volume of ethyl acetate. The solvent was removed by evaporation and the residue analysed by HPLC/MS. Peaks were observed with molecular mass m/z (M=H)=732 and 746 consistent with the expected masses for 13-isopropyl erythromycin B and 13-secbutylerythromycin B. Other erythromycins could be observed.

### EXAMPLE 7

Construction of S. avermitilis SGK-DELO/HP275

S. avermitilis strain SGK-DELO displays no antimicrobial activity (presumably due to loss of production of oligomycin). SGK-DELO was produced as follows. Spores of strain of S. avermitilis ATCC31272 were mutagenised conventionally using uv light. Dilutions of the spores were spread for single colonies, patched, and grown for 10 days before spotting M. luteus close to the patch, and growing for 24 hours at 30° C. Nineteen single colonies were chosen as they appeared to not inhibit the growth of the test organism. These colonies were picked using a glass pipette and plated onto YEME agar containing 100 μg/ml nalidixic acid. After three days growth the strains were replated onto a range of media including sporulation media to check for additional morphology changes. Further tests using liquid media were used to ensure antimicrobial activity was not present. Strain S. avermitilis SGK-DELO was chosen for further manipulation. This strain was transformed with demethylated (E. coli ET12567) pHP275, a derivative of pSet152 in which the desoamine biosynthesis genes DesI-VIII plus DesR from the pikromycin cluster have been inserted in tandem behind the ActI-ActII-Orf4promoter-activator system using the XbaI(methylated)-XbaI technology as described in WO 01/79520. Strains were transformed utilising standard techniques and selected on RM14 medium containing 100 μg/ml apramycin.

### EXAMPLE 8

Construction of S. avermitilis DELO/HP275+pSGK047 and its Use to Produce and Screen for Novel Erythromycins.

Plasmid pSGK047 was prepared from E. coli ET12567 to give demethylated DNA and used to transform S. avermitilis-DELO/HP275 utilising standard techniques. Thiostrepton resistant colonies were selected on RM14 medium containing 10 μg/ml thiostrepton. Individual thiostrepton resistant colonies were patched in 1 cm2 areas onto a large (25×25 cm) square agar plate grown for approximately 6 days to allow secondary metabolite production. At this stage small, plugs of agar were taken from each patch and placed at equally spaced intervals on a (25×25 cm) LB plate that had been poured with a culture of the indicator organism M. luteus. A patch of S. avermitilis DELO was used as a control. The LB plate was then placed at 4° C. for 4 hours to allow diffusion of natural products from plug to the LB agar before moving the plate 37° C. to allow growth of the screening organism. Individual plugs were screened by size of zone of inhibition.

The best producing colonies as judged by size of zone inhibition were used to verify the chemical nature of the active product. The strain was used to inoculate 6 ml YEME media and grown for 2 days at 30° C. 1.5 ml of this culture was used to inoculate YEME media and grown for a further 7 days. Cells were removed by centrifugation and the supernatant extracted three times with ethyl acetate. The extracts were combined and evaporated to dryness. HPLC-MS analysis of the extracts showed peaks corresponding to the expected masses of 5-O-desoaminyl-13-isopropyl-6-deoxyerythronolide, 5-O-desoaminyl-13-(sec butyl)-6-deoxyerythronolide, minor peaks including those with the mass expected for 5-O-desoaminyl-13-methyl-6-deoxyerythronolide and 5-O-desoaminyl-6-deoxyerythronolide could be observed.

### EXAMPLE 9

Construction of S. avermitilis SK-L

S. avermitilis SK-L is a derivative of S. avermitilis DELO in which the genes encoding AVES1 and AVES2 have been precisely deleted.

Plasmid pSGK246 is a pCJR24-based plasmid containing the 5′ end of the aves1 gene and aveC. Two synthetic oligonucleotides 5′-TCACGACATGGCGGGCGCGGCGAGGAAGGCC-3′ and 5′-TTTGCTAGCCTCGTCGGCCACTCCGAGGACCTCCCCTGCCG-3′ were used to amplify across aveF and aveD and 170 nt 5′ end of aves1. Two synthetic oligonucleotides were used to amplify across aveC and aveE and 40 nt of 3′ end of aves2, 5-TTTGCTAGCGAAACCGGACACACCACACACACGAAGGTG-3′ and 5-GTGATGTCCCAGTCGACGAGCAGCATTCCCG-3′. The amplified products were treated with kinase and digested overnight with NheI. The products were isolated by electrophoresis and purified from the gel. The two fragments were ligated together with pUC19 that had been digested with SmaI and treated with alkaline phosphatase as a three-way ligation. The ligation was transformed into electrocompetent E. coli DH10B and clones checked for the presence of the desired plasmid pSGK240. Plasmid pSGK240 was identified by extensive restriction digest and sequenced to ensure no errors had been introduced during amplification, and that the internal NheI site was correctly formed. Plasmid pSGK240 was digested with EcoRI and XbaI, and the ˜5 kb fragment isolated by gel electrophoresis and purified from the gel. This fragment was ligated with the ˜3.3 kb EcoRI-SpeI fragment of pCJR24 described previously. The ligation was transformed into electrocompetent E. coli DH10B and clones checked for the presence of the desired plasmid pSGK246 by restriction digest.

Plasmid pSGK246 was prepared from E. coli ET12567 to give demethylated DNA and used to transform S. avermitilis DELO utilising standard techniques. Thiostrepton resistant colonies were selected on RM14 medium containing 5 μg/ml thiostrepton. Colonies were checked by Southern hybridisation for those carrying the correct integration event. One colony was chosen to complete the gene replacement. Spores of this strain were inoculated into YEME media without thiostrepton and subcultured through four rounds of three days. At this point, cells were protoplasted using standard techniques and plated for single colonies on sporulation media. Approximately three thousand colonies were picked for thiostrepton sensitivity. Colonies displaying sensitivity were repicked to verify loss of marker and screened for the correct replacement event by Southern hybridisation. Southern blotting utilising other ave PKS and cluster probes was used to try verify the remainder of the cluster was complete since it has been noted that S. avermitilis readily deletes parts of the cluster during gene replacements made in this manner. One clone, designated S. avermitilis SK-L, was chosen for further transformation. This strain was grown to confirm loss of avermectin production by HPLC analysis.

### EXAMPLE 10

Construction of Plasmid pSGK375 and pSGK376

Plasmid pSGK375 is a pCJR24-based plasmid containing the gene encoding the first 2 proteins of the avermectin polyketide synthase (AVES1 and AVES2) and AveC. Plasmid pSGK376 is a pCJR29 (SCP2 based plasmid) containing the same genes.

Plasmid pSGK375 was constructed as follows: Two oligonucleotide primers were used to amplify DNA from the S. avermitilis AVES1 gene extending from nucleotide 91. 5′-CACAGCTCATATGCAGAGGATGGACGGCGG-3′ and 5′-TCCAAAGCGTCACCACGCGTGCGGCGT-3′. The design of the oligonucleotides incorporated a NdeI site at the start codon and extended across an internal MluI site. The amplified product was cloned into SmaI cut and dephosphorylated pUC18 and sequenced to ensure errors were not introduced during amplification. The amplified product was removed from pUC by digestion with NdeI and MluI, isolated by gel electrophoresis and purified from the gel (Fragment O). A second pair of oligonucleotides 5′-CCGACACGCACACGGACGCGTGCCTTGGCGGGAGC-3′ and 5′-CCCCTTCTCCGTCTAGACCGACCTGCCC-3′ were used to amplify DNA of the AVES2 and AveC extending from nucleotide 28023 to 31863 extending from an internal MluI site to beyond the end of AveC and incorporating an XbaI site. Again the amplified product was cloned into SmaI cut and dephosphorylated pUC18 and sequenced to ensure errors were not introduced during amplification. The amplified product was removed from pUC by digestion with MluI and XbaI isolated by gel electrophoresis and purified from the gel (Fragment P). Plasmid pCJR24 was digested with NdeI and XbaI, and isolated by gel electrophoresis and purified from the gel (Fragment Q). Fragments O, P and Q were ligated together using DNA ligase. The ligation mixture was used to transform electrocompetent E. coli DH10B and individual clones checked for the presence of the desired plasmid pSGK374. Plasmid pSGK374 was identified by restriction pattern.

Plasmid pSGK374 was digested with MluI and treated with alkaline phosphatase and ligated to a 27521 bp fragment produced by digesting cosmid cosAVE15-2 with MluI. The ligation mixture was used to transform electrocompetent E. coli DH10B and individual clones checked by restriction digest for the presence and correct orientation of the desired plasmid pSGK375.

Plasmid pSGK375 was digested with NdeI and XbaI to release a fragment of approximately 30 kB containing AVE1 and AVE2 and aveC. The fragments from this digest were dephosphorylated to stop religation and ligated to the SCP2 based plasmid pCJR29 that had been digested with the same enzymes and purified by gel electrophoresis. The ligation mixture was used to transform electrocompetent E. coli DH10B cells and a considerable number of individual clones checked for the presence of the desired plasmid pSGK376 by restriction digest. Extensive restriction digestion verified the construct.

### EXAMPLE 11

Construction of Plasmids pSGK390, pSGK391 and pSGK392

Plasmid pSGK390, pSGK391 and pSGK392 are pCJR24 based plasmids (ie non replicating) containing a hybrid polyketide synthase based on AVE1 and AVE2 which encode the loading module and modules 1 to 6 of the avermectin polyketide synthase. In plasmids pSGK390, pSGK391 and pSGK392 the reductive loop of module two has been replaced with the reductive loops of DEBS module 4 and RAPS module 1 and 13, respectively.

Plasmid pSGK381 is a pUC19 based plasmid containing the entire AVE1 polyketide synthase from nucleotides 101 to 12480. A new polylinker region for pUC19 was produced by slowly annealing two phosphorylated oligonucleotides together. 5′-TATGTTCGAAG-3′ and 5′-AATTCTTCGAACA3-. The design of the oligonucleotides incorporated a BstBI site and ‘sticky ends’ of NdeI and EcoRI to allow cloning. pUC19 was digested with NdeI and EcoRI and the large fragment isolated by gel electrophoresis and purified from the gel. This fragment was ligated together with the new polylinker region. The ligation mixture was used to transform E. coli DH10B cells and individual clones screened for the presence of the desired plasmid pSGK380 by restriction digest and sequence analysis to ensure the veracity of the inserted oligonucleotides.

Plasmid pSGK380 was digested with NdeI and BstBI and the large fragment isolated by gel electrophoresis and purified from the gel. Plasmid pSGK375 was digested with NdeI and BstBI to release a fragment of approximately 12 kB containing AVE1. This fragment was isolated by gel electrophoresis, purified from the gel, and ligated together with the pUC based fragment isolated from pSGK380. The ligation mixture was used to transform E. coli DH10B cells and individual clones screened for the presence of the desired plasmid pSGK381. The identity of plasmid pSGK381 was confirmed by extensive restriction analysis.

Two synthetic oligonucleotides 5′-CACGGCAGGTACCACGCAGGCGATCGCGGACACCGAACGGC-3′ and 5′-CCCTCTAGAGGTGGGGAGATCTAGGTGGGTGTGGGTGTGGGGTTGGTTGTCGTGGTGGG TGTA-3′ were used to amplify AVEI DNA from nucleotide 5860 to 9030 using pSGK375 as a template. The design of the oligonucleotides amplified across an SgfI site and incorporated KpnI and XbaI sites for cloning and introduced a BglII site at the beginning of the reductive loop. The amplified fragment was digested with KpnI and XbaI, isolated by gel electrophoresis, purified from the gel and ligated into pUC18 digested with the same enzymes. The ligation mixture was used to transform E. coli DH10B cells and individual clones screened for the presence of the desired plasmid pSGK382. A second pair of oligonucleotides 5′-TCTAGAGCCCGGCTAGCCGGCCAGACACACGAACAACAGC-3′ and 5′-TCCAAGCTTGCCCTGTTCGAACGTTTCCCAAGTGGTTTCG-3′ was used to amplify AVE DNA from nucleotide 11507 to 12490, the design of these oligonucleotides incorporated XbaI and HindIII sites for cloning and a NheI site at the end of the reductive loop. The amplified fragment was digested with HindIII and XbaI, isolated by gel electrophoresis, purified from the gel and ligated into the previous construct pSGK382, which had been digested with HindIII, and XbaI and isolated by gel electrophoresis and purified from the gel. The ligation mixture was used to transform E. coli DH10B cells and individual clones screened for the presence of the desired plasmid pSGK383. The identity of pSGK383 was confirmed by restriction analysis and DNA sequence analysis to confirm errors were not introduced during amplification.

Plasmid pSGK383 was digested with BglII and NheI and isolated by gel electrophoresis and the large fragment purified from the gel. Plasmids pJLK41, pJLK141 pJLK28 (See Patent GB99/02158) contain the reductive loop swaps from DEBS module 4 and RAPS module 1 and 13, respectively. These plasmids were digested with BglII and NheI and the ˜3 kb fragment isolated by gel electrophoresis, purified from the gel and each fragment ligated together with the fragment isolated from pSGK383. pJLK141 was treated as a partial digest due to an internal NheI site. Individual ligation mixtures were used to transform E. coli DH10B cells and individual clones screened for the presence of the desired plasmids pSGK384 and pSGK385 and pSGK386. The identities of these plasmids were confirmed by restriction analysis. Plasmids pSGK384 and pSGK385 and pSGK386 were digested with SgfI and BstBI and the fragments used to replace the equivalent fragment in pSGK381 by ligation. Individual ligation mixtures were used to transform E. coli DH10B cells and individual clones screened for the presence of the desired plasmids pSGK387 and pSGK388 and pSGK389. The identities of these plasmids were confirmed by restriction analysis. These plasmids contain the entire AVE1 with the reductive loop of module 2 replaced by a reductive loop DH-ER-KR.

To regenerate the bi-protein PKS, plasmids pSGK387, pSGK388 and pSGK389 were digested with NdeI and BstBI and the large fragment isolated by gel electrophoresis and purified from the gel. Plasmid pSGK375 was digested with NdeI and BstBI (care was taken to ensure complete digestion) and the larger fragment isolated by gel electrophoresis and purified from the gel. Significant care was taken during gel purification to avoid DNA shearing or irreversible binding to the purification resin. This fragment was ligated together with the fragments isolated from pSGK387, pSGK388 and pSGK389. The ligation mixtures were used to transform E. coli DH10B cells and individual clones screened for the presence of the desired plasmids pSGK390, pSGK391 and pSGK392. The identities of these plasmids were confirmed by extensive restriction analysis.

### EXAMPLE 12

### Construction of Plasmids pSGK393, pSGK394 and pSGK395

Plasmid pSGK393 pSGK394 and pSGK395 are pCJR29 based plasmids (ie replicating) containing a hybrid polyketide synthase based on AVE1 and AVE2 which encode the loading module and modules 1 to 6 of the avermectin polyketide synthase. In plasmid pSGK393, pSGK394 and pSGK395 the reductive loop of module two has been replaced with the reductive loops of DEBS module 4 and RAPS module 1 and 13, respectively.

To regenerate the replicating PKS plasmids pSGK387, pSGK388 and pSGK389 were digested with NdeI and BstBI and the large fragment isolated by gel electrophoresis and purified from the gel. Plasmid pSGK376 was digested with NdeI and BstBI (care was taken to ensure complete digestion) and the larger fragment isolated by gel electrophoresis and purified from the gel. Again significant care was taken during gel purification to avoid DNA shearing or irreversible binding to the purification resin. This fragment was ligated together with the fragments isolated from pSGK387, pSGK388 and pSGK389. The ligation mixtures were used to transform E. coli DH10B cells and individual clones screened for the presence of the desired plasmids pSGK393, pSGK394 and pSGK395 utilising the introduced sites. The identities of these plasmids were confirmed by restriction analysis.

### EXAMPLE 13

Construction of S. avermitilis SK-L/pSGK375, pSGK376, pSGK390, pSGK391, pSGK392, pSGK393, pSGK394, pSGK395 and Use and Screening for Anthelmintic Activity Due to the Production of Novel Avermectins.

Plasmids pSGK375, pSGK376, pSGK390, pSGK391, pSGK392, pSGK393, pSGK394, and pSGK395 were prepared from E. coli ET12567 to give demethylated DNA and used to transform S. avermitilis SK-L utilising standard techniques. Thiostrepton resistant colonies were selected on RM14 medium containing 5-10 μg/ml thiostrepton. Resistant colonies were replica plated onto microtitre plates for anthelmintic assay. S. avermitilis and S. avermitilis-SK-L was used as a control. Each well contained production media containing per litre Starch 80 g, Calcium carbonate 7 g, Pharmamedia 5 g, dipotassium hydrogen phosphate 1 g, magnesium sulfate 1 g, glutamic acid 0.6 g, ferrous sulphate heptahydrate 0.01 g, zinc sulfate 0.001 g, manganous sulfate 0.001 g, bactoagar 15 g. Final volume was adjusted to 1 litre with tapwater, pH adjusted to 7.2 and the media autocleaved at 121 C for 25 minutes before aliquoting to the microtitre plates. Replicas were grown at 30° C. for 2 weeks in a humidity controlled static incubator before assay. Anthelmintic activity against 1-week old Caenorhaditis elegans was evaluated directly on the agar plate using a modification of the screening test described by Simpkin and Coles in Parasitology, (1979) 79, 19. Extent of paralysis/death was assayed using a dissection microscope. This assay allowed us to assess whether the integration had occurred at the correct position without further rearrangement/amplification and had led to successful production of bioactive compound, allowing us to determine the most efficient reductive loop swap. Not all the transformants were producers. Subsequent screening of positive producing colonies by conventional HPLC confirmed production of the expected C-22-C23 dihydroavermectin series in addition to the normal avermectins by comparison with authentic standards.

### EXAMPLE 14

Production and Screening of a Library of Modified PKS for Novel Bioactive Polyketides

Malonyl-CoA specific extender ATs were amplified from a range of PKS of various organisms including S. fradiae, S. hygroscopicus, S. cinnamonensis, S. venezulae, S. coelicolor, S. avermitilis, and S. antibioticus. In each case genomic DNA or cosmids if available were used as a template. The design of the oligonucleotides for each extender incorporated MscI and AvrII sites exactly as illustrated in example 1 and Oliynyk et al. (Chem. Biol. (1996) 3, 833-839). Amplified products were digested with MscI and AvrII, isolated by gel electrophoresis, and the appropriate bands (˜1 kb) cut from the gel. In some cases it was clear that the bands were a mixture with the designed oligonucleotides binding at alternative sites. In a few cases this was confirmed by ligating the amplified product into SmaI cut pUC18 and sequencing the product, in some cases the product represented an AT from elsewhere in the cluster, in others the amplified AT was unknown. The amplified products were purified from the gel and normalised for concentration (˜10 ng/μl) before combining all of the amplified products to form a library of extender ATs. pHP012 was demethylated and digested with MscI and AvrII and isolated by gel electrophoresis and purified from the gel. This fragment (˜20 ng) was ligated together with 20 ng of the AT library. The entire ligation mixture (5 μl) was transformed into electrocompetent E. coli DH10B (5 transformations). In each case the entire transformation was spread onto LB plates (100 μl/plate), colonies were grown at 37° C. overnight. Colonies were harvested by pipetting 0.5 ml LB broth onto each plate and washing the colonies from the plate. DNA was prepared directly from these bacterial colonies, digested with SfiI and a ˜2.2 kb fragment (comprised of a library) isolated by gel electrophoresis and purified from the gel. This fragment was ligated with pHP010 that has been previously extensively digested with SfiI, treated with alkaline phosphatase and purified by phenol extraction and ethanol precipitation. Extensive controls were performed to ensure this vector was completely digested, and background colony formation was essentially zero. The ligation mixture was used to transform electrocompetent E. coli DH10B cells as before. It was important to recover the cells after transformation at room temperature for 2 hours and grow overnight at 30° C. Once colonies had grown to a reasonable size they were harvested as previously and combined. While preparing DNA directly from these colonies as previously was successful there was not necessarily sufficient DNA for transformation into Sacch. erythraea JC2 to give significant numbers of colonies. Hence it was necessary to amplify the library. Five flasks containing 100 ml LB+ampicillin were inoculated with 1 ml of combined culture washed from the plates and grown for about six hours. Cells were harvested and DNA prepared using Qiagen Tip100 columns. DNA was combined and quantitated by spectrophotometer. This DNA comprises a library of DEBS1+2+3 in which AT4 has been replaced by an alternative AT, all under the control of the act promoter.

Approximately 60 μg DNA was used to transform the protoplasts resulting from 30 ml S. erythraea JC2 growth when protoplasted under standard conditions and using standard techniques, care was taken to ensure minimal non-protoplasted cells remained, although false-positives (ie non-resistant colonies growing on thiostrepton-containing plates) due to these cells does not interfere with the biological screen as JC2 does not produce an antimicrobial substance. Transformed cells were plated onto multiple (40) R2T20 plates and recovered for 24 hours before overlaying with 40 μg/ml thiostrepton. These plates were grown at 30° C. by which time well-sporulating and in the main individual transformed colonies (approximately 50 per plate) were visible. Colonies were replica plated onto R2T20 containing 10 μg/ml thiostrepton and grown for a further seven days to ensure secondary metabolite production. These plates were overlayed with a 1 ml diluted culture of thiostrepton resistant B. subtilis before moving the plate 37° C. to allow growth of the screening organism. Colonies were screened by size of zone of inhibition to determine the most efficient AT swap. Ambiguities, particularly observed when the colonies were too close, were resolved by repatching from the replica plate and rescreening. After this screening step it was necessary to check the chemical nature of compounds produced to distinguish against those producing only natural erythromycins, rather than the desired 6-desmethyl compounds. We observed some production of natural erythromycin A that is probably due to a redundancy (ie mixture of extender ATs for malonyl- or methylmalonyl-CoA) in the specific AT library we used in this experiment.

### EXAMPLE 15

Plasmid pLSB136 is a pCJR24-based plasmid containing DEBS1, DEBS2 and DEBS3 under the control of the actI promoter. The AT domain of DEB1 (module 1) has been exchanged with the AT domain of rapamycin module 2. Plasmid pLSB136 was constructed by several intermediate plasmids as follows. Plasmid pCJR26 (Rowe et al. (1998) Gene 216, 215-223) containing the malonyl-CoA-specific AT domain from module 2 of the rapamycin PKS in place of the natural AT domain of module 1 of DEBS1-TE was digested with NdeI and BstBI to yield a 4596 bp fragment containing rap AT2. This fragment was ligated with a ˜19 kb fragment from pHP010 (see Example 1) that had been digested with NdeI and BstBI. The ligation mixture was used to transform electrocompetent E. coli DH10B cells and individual clones checked for the presence of the desired plasmid pLSB134. pHP010 was digested with BstBI and a ˜14.4 kb fragment isolated by gel electrophoresis and purified from the gel. This fragment was ligated with pLSB134 that has been digested with BstBI and treated with alkaline phosphatase. The ligation mixture was used to transform electrocompetent E. coli DH10B cells and the individual clones checked for the correct orientation of the BstBI fragment and hence for the presence of the desired final plasmid pLSB136.

### EXAMPLE 16

Construction of Sacch. erythraea NRRL2338 JC2 (pLSB136) and Screening it for Production of Novel Bioactive Erythromycins

Plasmid pLSB136 was used to transform Sacch. erythraea NRRL2338 JC2 protoplasts using standard techniques. Sacch. erythraea NRRL2338 JC2 has been precisely deleted of the entire DEBS1+2+3 leaving the TE as a homology region for integration of PKS plasmids (Rowe et al. (1998) Gene 216, 215-223). The transformation mixture was plated onto R2T20 plates and recovered for 24 hours before overlaying with 40 μg/ml thiostrepton. Colonies were grown for approximately 7-10 days to allow sporulation before harvesting the spores and plating dilutions onto R2T20 agar containing 40 μg/ml thiostrepton and grown for 7 days at 30° C. Individual thiostrepton resistant colonies were patched in 1 cm2 areas onto a large (25×25 cm) square R2T20 agar plate leaving reasonable space between each patch and grown for approximately 5 days to allow secondary metabolite production. At this stage small, plugs of agar were taken from each patch and placed at equally spaced intervals on a (25×25 cm) LB plate that had been poured with a culture of the indicator organism M. luteus. A patch of JC2 was used as a control. The LB plate was then placed at 4° C. for 4 hours to allow diffusion of natural products from plug to the LB agar before moving the plate 37° C. to allow growth of the screening organism. Individual plugs were screened by size of zone of inhibition. The production of active product confirmed to us that the vector construction had been correct, and particularly that the last step (BstBI fragment) had inserted in the correct orientation.

To verify the exact chemical nature of the active product identified by the screening S. erythraea NRRL 2338 (pLSB136) was used to inoculate 5 ml TSB containing 5 μg/ml thiostrepton. Of the isolates we choose the best producer as judged by the bioactivity. After three days growth 0.5 ml of this culture was used to inoculate 10 ml of EryP medium containing 5 μg/ml thiostrepton in a 25 ml flask. The flask was incubated at 30° C., 250 rpm for 6 days. At this time 1 ml supernatant was adjusted to ˜pH 9 with ammonia and extracted with an equal volume of ethyl acetate. The solvent was removed by evaporation and the residue analysed by HPLC/MS. A significant peak was observed with molecular mass m/z [M+H]+=704 for 12-desmethyl erythromycin B.

### EXAMPLE 17

The GPS-LS linker scanning system is marketed by New England Biolabs as a tool for generating 15 bp insertions by placing a transposon into target DNA at random locations in vitro. The kit is marketed as a tool to study protein structure “segments of protein sequence located at the surface of the three dimensional structure or in connector regions will frequently tolerate insertions of a few amino acids and remain functional, while segments that are buried, or part of an active site are generally intolerant of such insertions”. We believed that we could use this kit to generate a random library of erythromycin PKS containing 15 bp insertions in the DEBS genes. Of these insertions a certain subset would be in frame and result in a functional PKS, and a subset would possess insertions in the domains that made the domain non-functional while the entire PKS remained functional. Using the technology described in this patent it is possible to screen the library for modified PKS (i.e. containing an insertion) that still produced an active product. In the example described herein all the active but modified PKS examined by LCMS still produce erythromycin A rather than novel erythromycins, although some at much lower levels than normal. This experiment illustrates the ability of the PKS to tolerate insertions. However, it will be obvious to those skilled in the art that if we were to screen greater numbers of the PKS that are found to produce active products we would uncover PKS producing novel erythromycins. For example, erythromycins with altered oxidation state due to disruption (due to insertion) of ketoreductase functionality, or alternatively with altered side chains due to disruption (by insertion) of acyl transferase specificity.

The transposon library was generated following closely the protocol supplied. Target DNA was pHP010 (Example 1, a pCJR24 derivative containing DEBS1, DEBS2 and DEBS3 under the control of the act promoter). Approximately 100 ng target DNA was used in a 20 μl reaction (i.e. 2 μl GPS buffer, 30 ng pGPS5, 1 μl TnsABC* transposase, 1 μl start solution) at 37° C. After 1 hour the reaction was stopped by incubating at 75° C. The reaction mixture was transformed into electrocompetent E. coli DH10B and selected on LB Agar containing ampicillin (100 μg/ml) and kanamycin (20 μg/ml) and grown overnight at 30° C. Overnight cultures of these colonies were grown at 5 ml scale and DNA was prepared from each and digested with PmeI to remove the transposon. PmeI was inactivated at 65° C. and the reaction mixture diluted 5 fold and treated with DNA ligase to recircularise. The ligation mixtures were transformed into electrocompetent E. coli cells and plated onto LB plus ampicillin. Resistant colonies were checked for lack of a kanamycin marker by streaking onto LB containing ampicillin (100 μg/ml) and kanamycin (20 μg/ml). DNA from these colonies (˜2-5 μg each) was used to transform Sacch. erythraea NRRL2338 JC2 protoplasts using standard techniques. The transformation mixture was plated onto R2T20 plates and recovered for 24 hours before overlaying with 40 μg/ml thiostrepton. Colonies were grown for approximately 7-10 days to allow sporulation before harvesting the spores and plating dilutions onto R2T20 agar containing 40 μg/ml thiostrepton and grown for 7 days at 30° C. Individual thiostrepton resistant colonies were patched in 1 cm2 areas onto R2T20 agar and grown for approximately 5 days to allow secondary metabolite production. At this stage small, plugs of agar were taken from each patch and placed at equally spaced intervals on a (25×25 cm) LB plate that had been poured with a culture of the indicator organism M. luteus. The LB plate was then placed at 4° C. for 2 hours to allow diffusion of natural products from plug to the LB agar before moving the plate 37° C. to allow growth of the screening organism. This allowed identification of transposon insertion-containing PKS that maintained functionality (i.e. produced bioactive products), although a small number of the insertions could be contained in the vector (as opposed to PKS) system, many of these would result in a non-functional plasmid/promoter/resistance function and not be carried through to the end library. To verify the nature of the products produced by the PKS, small plugs of the some of the bioactive cultures were extracted using ethyl acetate (pH9), and analysed by LCMS. In each case the extract was shown to contain erythromycin A (rather than novel erythromycins, although only a very small proportion of the library was analysed). To confirm that the PKS had been modified, a sample of the some of the DNAs used to transform JC2 was sequenced using the primer N supplied in the kit. Approximately 70% gave sequence corresponding to that of the erythromycin PKS (For example, we obtained sequence corresponding to KS2, AT0, TE and KR2 implying the transposon was inserted in, or near to, these regions).

1. A method of producing a bioactive compound having a known bioactivity comprising:
a) providing host cells which substantially lack said bioactivity but possess at least one first enzymic activity or nucleic acid encoding at least one enzyme which is functional in a pathway leading to a compound having such bioactivity;
b) introducing into said host cells nucleic acid expressible in the host cells to provide at least one second enzymic activity; wherein said first and second activities together enable the transformed host cells to produce at least one screenable compound which may have said known bioactivity; and
c) screening for said bioactivity.

2. The method according to claim 1 wherein said step (c) is carried out on a culture of whole cells.

3. The method according to claim 1 wherein said step (c) is carried out on supernatant from the cultured cells.

4. The method according to claim 1 wherein said step (c) is carried out on a lysate or extract derived from the cells.

5. The method according to claim 1 wherein said bioactivity is a pharmacological activity.

6. The method according to claim 1 wherein said bioactivity is selected from the group consisting of antibacterial, antifungal, anticancer, antiviral, motilide, insecticidal, anthelmintic, herbicidal, anticoccidal, anticoagulant, anti-inflammatory, antiprotozoal, antiplatelet, anti-hypertensive, antiproliferative, proliferative, neuroregenerative, hair growth promoting, anti-fibrotic, antimalarial, antiplasmodial, antiangiogenesis, anticholesterol, cytotoxic, protein inhibition, protein synthesis inhibition, protein activation and immunosuppressant activities.

7. The method according to claim 1 further comprising:
(d) cultivating cells identified by the screening; and
(e) isolating said bioactive compound.

8. The method according to claim 1 wherein said introduction of nucleic acid into said host cells in step b) comprises introducing a multiplicity of different nucleic acid sequences into a corresponding multiplicity of host cells or host cell populations so that a proportion of the resulting transformed cells are thereby enabled to produce at least one compound having said bioactivity, the screening step leading to the identification of said cells.

9. The method of claim 8 wherein the multiplicity of transformed cells are enabled to produce a multiplicity of different bioactive compounds.

10. The method according to claim 8 further including a step of preparing said multiplicity of different nucleic sequences by
(a) providing a vector carrying at least one protein-encoding gene required for the biosynthesis of at least one bioactive substance; and
(b) genetically manipulating said at least one gene on said vector to produce a multiplicity of vectors carrying different manipulated versions of said one or more genes.

11. The method according to claim 1 wherein a single nucleic acid sequence is introduced into a multiplicity of cells containing or providing respective different first enzymic activities so that at least some of the cells are enabled to produce respective different active compounds.

12. The method according to claim 1 wherein said at least one first enzymic activity is an activity that contributes to a secondary metabolic pathway.

13. The method according to claim 1 wherein said at least one first enzymic activity and said at least one second enzymic activity comprise at least one activity selected from the croup consisting of glycosylation, methylation, oxidation, polyketide synthesis, isomerisation, ester formation, epimerisation, decarboxylation, lactonisation, acetylation or other acylation, amination, reduction dehydration, deoxysugar synthesis, and starter unit synthesis.

14. The method according to claim 1 wherein a first enzymic activity and a second enzymic activity comprise glycosylation and polyketide synthesis.

15. The method according to claim 1 wherein said at least one second enzymic activity leads to the production of a material which is a substrate for said at least one first enzymic activity, which leads to the production of a bioactive material.

16. The method according to claim 1 wherein said step of providing host cells includes a step of providing a precursor cell and genetically manipulating it to produce the host cell.

17. The method according to claim 1 wherein the bioactive compound is selected from the group consisting of polyketides, nonribosomal peptides, mixed polyketide-nonribosomal peptides, fatty acids, terpenes, alkaloids, aminoglycosides, shikimic acid derivatives, flavonoids, coumarins, polyglycosides, proteins, polysaccharides, flavones and other flavonoids, nitrogen containing compounds such as indoles and pyrroles, anthraquinones, lignans, coumarins, stilbenes, depsipeptides, peptides and nucleic acids, steroids and other hormones.

18. The method according to claim 17 wherein the bioactive compound is a polyketide.

19. The method according to claim 1 wherein said introduction of nucleic acids in step b) employs a vector containing PKS genes of a cluster or derivatives thereof; and said host cells are cells of a complementary but specifically prepared or selected host strain that possesses biosynthetic pathway enzymes which, when expressed together with the vector-encoded biosynthetic pathway genes, will give a desired bioactivity.

20. The method according to claim 19 wherein said PKS genes of said vector have undergone genetic manipulation.

21. The method according to claim 19 wherein said enzymes of the host cells have undergone genetic manipulation.

22. Recombinant cells produced by introducing nucleic acids into host cells which substantially lack a predetermined bioactivity but possess at least one first enzymic activity or nucleic acid encoding at least one enzyme which is functional in a pathway leading to such bioactivity; wherein said introduced nucleic acids are expressible in the host cells to provide at least one second enzymic activity; and wherein said first and second activities together enable the host cells to produce at least one compound suspected of having a known bioactivity.

23. The recombinant cells according to claim 22 wherein the host cells are selected from the group consisting of prokaryotic and eukaryotic microorganisms and cells of higher eukaryotes.

24. The recombinant cells according to claim 22 wherein the host cells are selected from the group consisting of prokaryotic, fungal and mammalian cells.

25. The recombinant cells according to claim 22 wherein the host cells are actinomycete cells.

26. The recombinant cells according to claim 25 wherein the host cells are selected from the group consisting of Saccharopolyspora erythraea, Streptomyces coelicolor, Streptomyces avermitilis, Streptomyces griseofuscus, Streptomyces cinnamonensis, Streptomyces fradiae, Streptomyces eurythermus, Streptomyces longisporoflavus, Streptomyces hygroscopicus, Saccharopolyspora spinosa, Micromonospora griseorubida, Streptomyces lasaliensis, Streptomyces venezuelae, Streptomyces antibioticus, Streptomyces lividans, Streptomyces rimosus, Streptomyces albus, Streptomyces rochei, Actinoplanes Sp., Amycolatopsis mediterranei, Nocardia Sp. and Streptomyces tsukubaensis.

27. The method according to claim 1 wherein said at least one first enzymic activity comprises at least one activity selected from the group consisting of glycosylation, methylation, oxidation, polyketide synthesis, isomerisation, ester formation, epimerisation, decarboxylation, lactonisation, acetylation or other acylation, amination, reduction dehydration, deoxysugar synthesis, and starter unit synthesis.

28. The method according to claim 1 wherein said at least one second enzymic activity comprise at least one activity selected from the group consisting of glycosylation, methylation, oxidation, polyketide synthesis, isomerisation, ester formation, epimerisation, decarboxylation, lactonisation, acetylation or other acylation, amination, reduction dehydration, deoxysugar synthesis, and starter unit synthesis.
